(19) United States
(12) Patent Application Publication
(10) Pub. No.: US 2022/0208386 A1
(43) Pub. Date: Jun. 30, 2022

(54) DIAGNOSTIC FOR MATERNAL RISK OF HAVING A CHILD WITH AUTISM SPECTRUM DISORDER

(71) Applicants: Arizona Board of Regents on Behalf of Arizona State University, Tempe, AZ (US); Rensselaer Polytechnic Institute, Troy, NY (US)

(72) Inventors: JAMES B. ADAMS, Tempe, AZ (US); JUERGEN HAHN, Troy, NY (US)

(21) Appl. No.: 17/601,582

(22) PCT Filed: Apr. 6, 2020
(86) PCT No.: PCT/US2020/026915

§ 371 Date: Oct. 5, 2021

Related U.S. Application Data
(60) Provisional application No. 62/830,037, filed on Apr. 5, 2019.

(51) Int. Cl. G16H 50/30; G01N 1/30; G01N 33/68; G01N 30/72; G16H 20/10; G16H 20/40; G16H 20/70

(52) U.S. Cl. CPC G16H 50/30; G01N 1/30; G01N 33/68; G16H 20/70; G16H 20/10; G16H 20/40; G01N 30/7233

(57) ABSTRACT

Provided herein are methods of obtaining and applying measurements of metabolites to quantifying maternal risk of having a child with autism spectrum disorder (ASD), with high specificity and sensitivity.

### CROSS-REFERENCE TO RELATED APPLICATIONS

This application claims the benefit of U.S. Provisional Patent Application No. 62/830,037 filed Apr. 5, 2019, the entire disclosure of which is incorporated herein by reference.

### FIELD OF THE INVENTION

The present disclosure generally relates to specific and sensitive methods for early detection of autism spectrum disorder (ASD) in a child, and more particularly to methods of identifying mothers at risk of bearing a child with ASD.

### BACKGROUND

The diagnosis of autism spectrum disorder (ASD) is currently based on assessment of behavioral symptoms in patients considered to be at risk. Such symptoms include major impairments in social communication and skills, stereotyped motor behaviors, and tightly focused intellectual interests. Strong evidence exists that the underlying causes of ASD are present in earliest infancy and even prenatally, and involve a complex interaction of genetic and environmental factors. Yet diagnosis of ASD at early ages is extremely difficult because some symptoms are simply not present in early infancy and other symptoms are difficult to distinguish from normal development. One national prevalence study of eight-year-olds with ASD found that the median age of diagnosis was 46 months for autism and 52 months for ASD; however, this study did not account for children and adults diagnosed at ages above eight years, so the true median age of diagnosis is even higher. Stable diagnoses of ASD have been found in children as young as 18 months, representing a significant disconnect between current and ideal outcomes.

At the same time, early diagnosis is important because available interventions are most effective if started early in life. A number of different intervention models have been demonstrated to be significantly helpful for many children with ASD, such as the Early Start Denver Model which has been found effective when started in early infancy. Early intervention may maximize the opportunity for improving neural connectivity while brain plasticity is still high, likely helping to reduce the severity of ASD or even prevent it from fully manifesting.

Even though ASD is currently diagnosed solely based upon clinical observations of children, certain physiological factors are believed to contribute or be affected by ASD. Development of a biomarker-based test for ASD, using quantifiable measures rather than qualitative judgement, could assist with screening for and diagnosing ASD earlier in childhood. This, in turn, would indicate if further evaluation is needed and allow for intervention and/or therapy to begin as early as possible. The value of ASD-related biomarkers goes beyond diagnosis, as they also offer the potential to evaluate treatment efficacy. This would serve as a complement to current behavioral and symptom assessments and help to further elucidate the underlying biological mechanisms affecting ASD symptoms. For example, multivariate statistical analysis of changes in plasma metabolites has been found to offer value for modeling changes in metabolic profiles and adaptive behavior resulting from clinical intervention. Functional neuroimaging biomarkers may also be promising indicators of biological response to treatment. In addition, eye-tracking metrics could represent further avenues for quantifying changes in behavior resulting from intervention and clinical trials. As with diagnostic biomarkers, such approaches can help to mitigate subjectivity in treatment assessment arising from the use of purely behavioral measures.

A need thus exists for efficient and reliable methods of early, and if possible prenatal diagnosis of ASD in children, to indicate early intervention to prevent ASD and/or to reduce the severity of symptoms.

### SUMMARY OF THE INVENTION

One aspect of the present disclosure encompasses a method for determining maternal risk of a female subject bearing a child with Autism Spectrum Disorder (ASD). The method comprises measuring the level of one or a combination of two or more metabolites selected from the metabolites listed in Table 1, Table 9, and Table 10 in a biological sample obtained from the subject. A level of the one or combination of metabolites in the biological sample significantly different from the level of the one or combination of metabolites in a control panel of metabolite levels is indicative of a risk of having a child with ASD. The risk can be determined pre-conception, during pregnancy, or after giving birth to the child. The age of the child after birth can range from about 1 day to about 10 years. The method can further comprise assigning a personalized medical, behavioral, or nutritional treatment protocol to the female subject before conception or giving birth. The method can further comprise assigning a personalized medical, behavioral, or nutritional treatment protocol to the child after birth.

The one or more metabolites are measured by preparing a sample extract and using Ultrahigh Performance Liquid Chromatography-Tandem Mass Spectroscopy (UPLC-MS/MS) to obtain the levels of the one or the combination of two or more metabolites in the reconstituted sample extract. The sample extract can be prepared by subjecting the sample to methanol extraction, and a dried sample extract can prepared from the methanol extraction. If a sample extract is dried, the dried sample extract is reconstituted for measuring the level of the one or combination of two or more metabolites. The method can further comprise removing protein from the biological sample.

A significantly different level of the one or combination of metabolites can be determined by applying each of the measured levels of the metabolites against a control panel of metabolite levels created by measuring metabolite levels of the one or combination of metabolites in control subjects with no history of bearing a child with Autism Spectrum Disorder (ASD). The panel can be stored on a computer system.

When the level of one metabolite is measured, applying each of the measured levels of the metabolites can comprise comparing the measured level of the metabolite in the sample to the level of the metabolite in the control panel of metabolite levels using a statistical analysis method selected from the standard Student t-test, the Welch test, the Mann-Whitney U test, the Welch t-test, and combinations thereof; and calculating the false discovery rates (FDR; calculates the p-value) and optionally the false positive rate (FPR; calculates the q-value) for the metabolite. A p-value of less than or about 0.05 and an FDR value of less than or about 0.1, is indicative of a risk of having a child with ASD.

When the levels of a combination of two or more metabolites are measured, applying comprises calculating the Type I (FPR; false positive rate) and Type II (FNR; false negative rate) errors for the combination of metabolites using FDA or logistic regression. A Type I error of about or below 10% and a Type II error of about or below 10% is indicative of a risk of having a child with ASD.

Another aspect of the present disclosure encompasses a method for determining increased maternal risk of a female subject bearing a child with ASD. The method comprises obtaining or having obtained a biological sample from the female subject; subjecting the sample to methanol extraction; drying the sample extract; reconstituting the sample extract; and measuring the level of one or a combination of two or more metabolites selected from the metabolites listed in Table 1, Table 9, and Table 10 in the reconstituted sample extract using Ultrahigh Performance Liquid Chromatography-Tandem Mass Spectroscopy (UHPLC-MS/MS). The method further comprises applying each of the measured levels of the metabolites against a control panel of metabolite levels created by measuring metabolite levels of the one or combination of metabolites in control subjects with no history of bearing a child with ASD, wherein the panel is stored on a computer system. The method can further comprising removing protein from the biological sample.

When the level of one metabolite is measured, applying comprises comparing the measured level of the metabolite in the sample to the level of the metabolite in the control panel of metabolite levels using a statistical analysis method selected from the standard Student t-test, the Welch test, the Mann-Whitney U test, the Welch t-test, and combinations thereof; and calculating the false discovery rates (FDR; calculates the p-value) and optionally the false positive rate (FPR; calculates the q-value) for the metabolite. A p-value of less than or about 0.05 and an FDR value of less than or about 0.1, is indicative of a risk of having a child with ASD.

When the levels of a combination of two or more metabolites are measured, applying comprises calculating the Type I (FPR; false positive rate) and Type II (FNR; false negative rate) errors for the combination of metabolites using FDA or logistic regression. A Type I error of about or below 10% and a Type II error of about or below 10% is indicative of a risk of having a child with ASD.

In any of the aspects described above, the biological sample can comprise any one of synovial, whole blood, blood plasma, serum, urine, breast milk, and saliva. Further, the biological sample can comprise cells. In some aspects, the biological sample is whole blood. Further, the level of a metabolite can be measured using reverse phase chromatography positive ionization methods optimized for hydrophilic compounds (LC/MS Pos Polar); reverse phase chromatography positive ionization methods optimized for hydrophobic compounds (LC/MS Pos Lipid); reverse phase chromatography with negative ionization conditions (LC/MS Neg); a HILIC chromatography method coupled to negative (LC/MS Polar); or combinations thereof.

The level of a metabolite can be calculated from a peak area and standard calibration curve obtained for the metabolite using the UPLC-MS/MS. Additionally, measuring metabolites can further include identifying each metabolite by automated comparison of the ion features in the sample extract to a reference library of chemical standard entries that included retention time, molecular weight (m/z), preferred adducts, and in-source fragments as well as associated MS spectra. The method can also further comprise calculating the area under the curve (AUC) of the receiver operating characteristic (ROC) curve for each metabolite. When the levels of a combination of two or more metabolites are measured, a multivariate analysis can further be combined with leave-one-out cross-validation to analyze the success of the model on classification. In any of the aspects described above, the risk of a female subject bearing a child with ASD can be determined pre-conception, during pregnancy, or after giving birth to the child.

The level of one metabolite can be measured to determine the risk of bearing a child ASD. The one metabolite can be selected from the metabolites listed in Table 2 and Table 10. In some aspects, the metabolite is Histidylglutamate or N-acetylasparagine.

The level of a combination of two metabolites can be measured to determine the risk of bearing a child ASD. The two metabolites can be selected from the combinations of metabolites listed in Table 3 and Table 14. In some aspects, the two metabolites are N-acetylasparagine and X-12680. In other aspects, the two metabolites are Histidylglutamate and 6-hydroxyindoel sulfate.

The level of a combination of three metabolites can be measured to determine the risk of bearing a child ASD. The three metabolites can be selected from the combinations of metabolites listed in Table 4 and Table 14. In some aspects, the three metabolites are 6-hydroxyindole sulfate, histidylglutamate, and N-acetylasparagine. In other aspects, the three metabolites are 6-hydroxyindole sulfate, histidylglutamate, and N-acetylasparagine. In yet other aspects, the three metabolites are histidylglutamate, N-acetylasparagine, and X-21310. In additional aspects, the three metabolites are 3-indoxyl sulfate, histidylglutamate, and N-acetylasparagine. In some aspects, the three metabolites are Histidylglutamate, N-formylanthranilic acid, and palmitoylcarnitine (C16).

The level of a combination of four metabolites can be measured to determine the risk of bearing a child ASD. The four metabolites can be selected from the combination of metabolites in Table 5 and Table 14. In some aspects, the four metabolites are Histidylglutamate, S-1-pyrroline-5-carboxylate, N-acetyl-2-aminooctanoate*, and 5-methylthioadenosine (MTA).

The level of a combination of five metabolites can be measured. The five metabolites can be selected from the combination of metabolites in Table 6 and Table 15. In some aspects, the five metabolites are Glu-Cys, histidylglutamate, cinnamoylglycine, proline, and adrenoylcarnitine (C22:4)*. When the metabolites are Glu-Cys, histidylglutamate, cinnamoylglycine, proline, and adrenoylcarnitine (C22:4)*, each metabolite represents a group of metabolites correlated with the metabolite. The metabolites correlated with each metabolite can be as listed in Table 16. In the methods, the levels of metabolites correlated with each metabolite can also be measured.

The method can determine the maternal risk of bearing a child with ASD with a sensitivity of at least about 80% to 90%, a specificity of at least about 80% to 90%, or both. The method can also determine the maternal risk of bearing a child with ASD with a misclassification error of about 5% or less, such as about 3%. Further, the method can determine the maternal risk of bearing a child with ASD with an accuracy of about 95% or more, such as with approximately 97% accuracy.

The method can further comprise assigning a medical, behavioral, and/or nutritional treatment protocol to the subject when the subject is at increased risk of bearing a child with ASD. A treatment protocol can be personalized to the subject. For instance, a treatment protocol can be personalized based on the metabolites found to be significantly different in a sample obtained from the subject when compared to a control and identified using the method described herein. Such a personalized treatment protocol can include adjusting in the subject the level of the one or a combination of two or more metabolites found to be significantly different in a sample obtained from the subject. The treatment protocol can also include adjusting the levels of one or more metabolite associated with the one or combination of two or more metabolites identified as having a level in the biological sample significantly different from the level of the one or combination of metabolites in the control sample. In some aspects, the treatment protocol comprises supplementation with vitamin B12, folate, or combination thereof before and/or during pregnancy.

Yet another aspect of the present disclosure encompasses a method of determining a personalized treatment protocol for a pregnant subject or a subject contemplating conception and at risk of having a child with ASD. The method comprises measuring in a biological sample obtained from the subject the level of one or combination of two or more metabolites selected from the metabolites listed in Table 1, Table 9, and Table 10 and any combination thereof, identifying one or a combination of metabolites having a level in the biological sample significantly different from the level of the one or combination of metabolites in a control sample, and assigning a personalized medical, behavioral, or nutritional treatment protocol to the subject, wherein a level of the one or combination of metabolites in the biological sample significantly different from the level of the one or combination of metabolites in a control sample is indicative of a risk of having a child with ASD.

Another aspect of the present disclosure encompasses a method of monitoring the therapeutic effect of an ASD treatment protocol in a pregnant subject or a subject contemplating conception and at risk of having a child with ASD. The method comprises measuring in a first biological sample obtained from the subject the level of one or a combination of metabolites selected from the metabolites listed in Table 1, Table 9, and Table 10 and any combination thereof, measuring in a second biological sample obtained from the subject the level of the one or combination of metabolites, and comparing the level of the one or combination of metabolites in the first sample and the second sample, wherein maintenance of the level of the one or combination of metabolites or a change of the level of the one or combination of metabolites to a level of the one or combination of metabolites in a control sample is indicative that the treatment protocol is therapeutically effective in the subject.

One aspect of the present disclosure encompasses a kit for performing any of the methods described above. The kit comprises a container for collecting the biological sample from the subject and solutions and solvents for preparing an extract from a biological sample obtained from the subject. The kit further comprises instructions for (i) preparing the extract, (ii) measuring the level of one or more metabolites selected from the metabolites listed in Table 1, Table 9, and Table 10 using Ultrahigh Performance Liquid Chromatography-Tandem Mass Spectroscopy (UPLC-MS/MS); and (iii) applying the measured metabolite levels against a control panel of metabolite levels created by measuring metabolite levels of the one or combination of metabolites in control subjects with no history of bearing a child with ASD.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1. Preparation of client-specific technical replicates. A small aliquot of each client sample (colored cylinders) is pooled to create a CMTRX technical replicate sample (cylinder), which is then injected periodically throughout the platform run. Variability among consistently detected biochemicals can be used to calculate an estimate of overall process and platform variability.

FIG. 2. Visualization of data normalization steps for a multiday platform run.

FIG. 3. Scatter plot of the probabilities of being classified into one group or the other using a combination of variables from the FOCM/TS pathways, the additional measurements, and the top 50 metabolites from the metabolon.

### DETAILED DESCRIPTION

The present disclosure is based in part on the surprising discovery of metabolite biomarkers measured in a female subject and methods of using the biomarkers to determine, with a high level of sensitivity and specificity, the risk of the subject bearing a child with Autism Spectrum Disorder (ASD). The metabolites can be used to differentiate between mothers of young children with ASD (ASD-M) and mothers of young typically developing children (TD-M), for early detection of ASD in a child. In other words, a child can be diagnosed with ASD by measuring the metabolites in the mother. The biomarkers can be used to detect ASD shortly after the child is born, or even during pregnancy of the mother or before conception.

### I. Methods

One aspect of the present disclosure provides a method of determining maternal risk of a female subject bearing a child with ASD. The method comprises measuring the level of metabolites in a maternal biological sample obtained from the subject. The female subject can be, without limitation, a human, a non-human primate, a mouse, a rat, a guinea pig, and a dog. In some aspects, the subject is a human female. The risk of bearing a child with ASD can be determined pre-conception, during pregnancy, or after giving birth to the child.

A sample may include but is not limited to, a cell, a cellular organelle, an organ, a tissue, a tissue extract, a biofluid, or an entire organism. The sample may be a heterogeneous or homogeneous population of cells or tissues. As such, metabolite levels or concentrations can be measured within cells, tissues, organs, or other biological samples obtained from the subject. For instance, the biological sample can be bone marrow extract, whole blood, blood plasma, serum, peripheral blood, urine, phlegm, synovial fluid, milk, saliva, mucus, sputum, exudates, cerebrospinal fluid, intestinal fluid, cell suspensions, tissue digests, tumor cell containing cell suspensions, cell suspensions, and cell culture fluid which may or may not contain additional substances (e.g., anticoagulants to prevent clotting). The sample can comprise cells or can be cell free. Samples that include cells comprises metabolites that exist primarily inside of cells as well as those that primarily exist outside of cells. In some aspects, the sample comprises cells. In one aspect, the sample is whole blood.

In some aspects, multiple biological samples may be obtained for diagnosis by the methods of the present invention, e.g., at the same or different times. A sample, or samples obtained at the same or different times, can be stored and/or analyzed by different methods.

Methods for obtaining and extracting the metabolome from a wide range of biological samples, including cell cultures, urine, blood/serum, and both animal- and plant-derived tissues are known in the art. Although these protocols are readily available, the variable stability of metabolites and the source of a sample means that even minor changes in procedure can have a major impact on the observed metabolome. For instance, the fast turnover rate of enzymes and the variable temperature and chemical stability of metabolites require that metabolomics samples be collected quickly and handled uniformly and that all enzymatic activity be rapidly quenched in order to minimize biologically irrelevant deviations between samples that may result from the processing protocol.

A metabolomics extraction protocol can focus on a subset of metabolites (for example, water-soluble metabolites or lipids). Furthermore, an extraction protocol may focus on either a highly reproducible and quantitative extraction of a restricted set of metabolites (that is, targeted metabolomics) or the global collection of all possible metabolites (that is, untargeted metabolomics).

In some aspects, sample extracts are prepared by subjecting the sample to methanol extraction to remove proteins, dissociate small molecules bound to protein or trapped in the precipitated protein matrix, and to recover chemically diverse metabolites. In one aspect, a dried sample extract is prepared from the methanol extraction. A dried sample can then be reconstituted in a solvent for measuring the level of the one or combination of two or more metabolites.

Methods of measuring metabolites in a sample are known in the art. The methods can and will vary depending on the metabolites, the number of metabolites to be measured, and the biological sample in which the metabolites are measured, among other variables, and can be determined experimentally. Non-limiting examples of analytical techniques suitable for measuring metabolites include liquid chromatography-mass spectrometry (LC-MS), gas chromatography-mass spectrometry (GC-MS), nuclear magnetic resonance (NMR), enzyme assays, and variations on these methods. In some aspects, the metabolites are measured using Ultrahigh Performance Liquid Chromatography-Tandem Mass Spectroscopy (UPLC-MS/MS).

In some aspects, a sample extract is subjected to one or more than one measurement. For instance, a sample can be divided into more than one aliquot to measure metabolites using more than one analytical method. In some aspects, the level of metabolites in aliquots of the sample extract are measured using reverse phase chromatography positive ionization methods optimized for hydrophilic compounds (LC/MS Pos Polar); reverse phase chromatography positive ionization methods opti-mized for hydrophobic compounds (LC/MS Pos Lipid); reverse phase chromatography with negative ionization conditions (LC/MS Neg); and a HILIC chromatography method coupled to negative (LC/MS Polar).

The level of a metabolite can be determined from a peak area and standard calibration curve obtained for the metabolite using the UPLC-MS/MS. Additionally, measuring metabolites can further include identifying each metabolite such as by automated comparison of the ion features in the sample extract to a reference library of chemical standard entries that include retention time, molecular weight (m/z), preferred adducts, and in-source fragments as well as associated MS spectra.

The method comprises measuring the level of one, or a combination of two or more metabolites in the sample. For instance, the level of one or the levels of 2, 3, 4, 5, 6, 7, 8, 9, 10, 15, 20, 25, 30, 40, 50, 60 or more metabolites can be measured. The metabolites and combinations of metabolites can be selected from the metabolites listed in Table 1, Table 9, and Table 10.

A level of the measured one or combination of metabolites in the biological sample significantly different from the level of the one or combination of metabolites in a control panel of metabolite levels is indicative of a risk of having a child with ASD. A significantly different level of the one or combination of metabolites can be determined by applying each of the measured levels of the metabolites against a control panel of metabolite levels created by measuring metabolite levels of the one or combination of metabolites in control subjects with no history of bearing a child with ASD. The panel can be stored on a computer system. It is noted that a significant difference in the level of the metabolite can be an increase or a decrease in the level of the metabolite in the sample when compared to the level of the metabolite in the control panel of metabolite levels. The method can also further comprise calculating the area under the curve (AUC) of the receiver operating characteristic (ROC) curve for each metabolite. When the levels of a combination of two or more metabolites are measured, a multivariate analysis can further be combined with leave-one-out cross-validation to analyze the success of the model on classification. In any of the aspects described above, the risk of a female subject bearing a child with ASD can be determined pre-conception, during pregnancy, or after giving birth to the child.

In some aspects, the level of one metabolite is measured. When the level of one metabolite is measured, applying each of the measured levels of the metabolites can comprise comparing the measured level of the metabolite in the sample to the level of the metabolite in the control panel of metabolite levels using a statistical analysis method. Non-limiting examples of statistical analysis methods suitable for use when one metabolite is measured include analysis of variance (ANOVA), chi-squared test, correlation, factor analysis, Mann-Whitney U, Mean square weighted deviation (MSWD), Pearson product-moment correlation coefficient, regression analysis, Spearman's rank correlation coefficient, Student's t-test, Time series analysis, and Conjoint Analysis, among others, and combinations thereof. In some aspects, when the level of one metabolite is measured, applying each of the measured levels of the metabolites can comprise comparing the measured level of the metabolite in the sample to the level of the metabolite in the control panel of metabolite levels using a statistical analysis method selected from the standard Student t-test, the Welch test, the Mann-Whitney U test, the Welch t-test, and combinations thereof; and calculating the false discovery rates (FDR; calculates the p-value) and optionally the false positive rate (FPR; calculates the q-value) for the metabolite. In some aspects, a p-value of less than or about 0.05 and an FDR value of less than or about 0.1, is indicative of a risk of bearing a child with ASD.

When the level of one metabolite is measured to determine the risk of bearing a child ASD, the one metabolite can be selected from the metabolites listed in Table 2 and Table 10. In some aspects, the metabolite is Histidylglutamate or N-acetylasparagine. When the metabolite is Histidylglutamate or N-acetylasparagine

When the levels of a combination of two or more metabolites are measured, applying each of the measured levels of the metabolites against a control panel of metabolite levels created by measuring metabolite levels of the one or combination of metabolites in control subjects with no history of bearing a child with ASD comprises calculating the Type I (FPR; false positive rate) and Type II (FNR; false negative rate) errors for the combination of metabolites using FDA or logistic regression. A Type I error of about or below 25, 20, 15, or 10% and a Type II error of about or below 25, 20, 15, or 10% is indicative of a risk of having a child with ASD.

In some aspects, the level of a combination of two metabolites are measured to determine the risk of bearing a child having ASD. The two metabolites can be selected from the combinations of metabolites listed in Table 3 and Table 14. In some aspects, the two metabolites are N-acetylasparagine and X-12680. In other aspects, the two metabolites are Histidylglutamate and 6-hydroxyindoel sulfate.

The level of a combination of three metabolites can be measured to determine the risk of bearing a child ASD. The three metabolites can be selected from the combinations of metabolites listed in Table 4 and Table 14. In some aspects, the three metabolites are 6-hydroxyindole sulfate, histidylglutamate, and N-acetylasparagine. In other aspects, the three metabolites are 6-hydroxyindole sulfate, histidylglutamate, and N-acetylasparagine. In yet other aspects, the three metabolites are histidylglutamate, N-acetylasparagine, and X-21310. In additional aspects, the three metabolites are 3-indoxyl sulfate, histidylglutamate, and N-acetylasparagine. In some aspects, the three metabolites are Histidylglutamate, N-formylanthranilic acid, and palmitoylcarnitine (C16).

The level of a combination of four metabolites can be measured to determine the risk of bearing a child ASD. The four metabolites can be selected from the combination of metabolites in Table 5 and Table 14. In some aspects, the four metabolites are Histidylglutamate, S-1-pyrroline-5-carboxylate, N-acetyl-2-aminooctanoate*, and 5-methylthioadenosine (MTA).

The level of a combination of five metabolites can be measured. The five metabolites can be selected from the combination of metabolites in Table 6 and Table 15. In some aspects, the five metabolites are Glu-Cys, histidylglutamate, cinnamoylglycine, proline, and adrenoylcarnitine (C22:4)*. In some aspects, when the metabolites are Glu-Cys, histidylglutamate, cinnamoylglycine, proline, and adrenoylcarnitine (C22:4)*, each metabolite represents a group of metabolites correlated with the metabolite. The metabolites correlated with each metabolite can be as listed in Table 16. In the methods, the levels of metabolites correlated with each metabolite can also be measured.

Further, more than one combination of metabolites can be used to further improve the accuracy of an ASD diagnosis, including improving specificity and sensitivity, and reducing misclassification errors. For instance, the diagnosis obtained from a measurement of a combination of two metabolites in a whole blood sample can be combined with results from a combination of three metabolites measured in the sample to improve accuracy of a diagnosis.

Further, each metabolite can represent a group of metabolites correlated with the metabolite. In the methods, the levels of metabolites correlated with each metabolite can also be measured.

The method can determine the maternal risk of bearing a child with ASD with a high level of sensitivity. For instance, the method can determine the maternal risk of bearing a child with ASD with a sensitivity greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, or greater than or equal to 99%. The method can also determine the maternal risk of bearing a child with ASD with a high level of specificity. For instance, the method can determine the maternal risk of bearing a child with ASD with a specificity greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, or greater than or equal to 99%. In some aspects, the method can determine the maternal risk of bearing a child with ASD with a sensitivity of at least about 80% to 90%, a specificity of at least about 80% to 90%, or both.

The method can also determine the maternal risk of bearing a child with ASD with a low misclassification error, such as a misclassification error of about 10, 8, 9, 7, 6, 5, 4, 3, 2, 1% or lower. In some aspects, the method can also determine the maternal risk of bearing a child with ASD with a misclassification error of about 5% or less, or about 3% or less.

Further, the method can determine the maternal risk of bearing a child with ASD with an accuracy of about 75, 80, 85, 90, 95% or higher. In some aspects, the method can also determine the maternal risk of bearing a child with ASD with an accuracy of about 95% or higher, such as with an accuracy of about 97% or higher.

The method can further comprise assigning a medical, behavioral, and/or nutritional treatment protocol to the subject when the subject is at increased risk of bearing a child with ASD. A treatment protocol can also be assigned to a child born to a subject determined to be at high risk of having a child with ASD. Non-limiting examples of treatment protocols include behavioral management therapy, cognitive behavior therapy, early intervention, educational and school-based therapies, joint attention therapy, medication treatment, nutritional therapy, occupational therapy, parent-mediated therapy, physical therapy, social skills training, speech-language therapy, and combinations thereof. Non-limiting examples of medication treatment include antipsychotic drugs, such as risperidone and aripripazole, for treating irritability associated with ASD, Selective serotonin re-uptake inhibitors (SSRIs), tricyclics, psychoactive or anti-psychotic medications, stimulants, anti-anxiety medications, anticonvulsants, and Microbiota Transfer Therapy (MTT). In one aspect, the treatment protocol assigned to the child is MTT. MTT treatment methods are known in the art and generally relate to transferring beneficial fecal bacteria to replace, restore, or rebalance the ASD patient's gut microbiota.

When the level of one metabolite is measured, applying comprises comparing the measured level of the metabolite in the sample to the level of the metabolite in the control panel of metabolite levels using a statistical analysis method selected from the standard Student t-test, the Welch test, the Mann-Whitney U test, the Welch t-test, and combinations thereof; and calculating the false discovery rates (FDR; calculates the p-value) and optionally the false positive rate (FPR; calculates the q-value) for the metabolite. A p-value of less than about 0.05 and an FDR value of less than or about 0.1, is indicative of a risk of having a child with ASD.

When the levels of a combination of two or more metabolites are measured, applying comprises calculating the Type I (FPR; false positive rate) and Type II (FNR; false negative rate) errors for the combination of metabolites using FDA or logistic regression. A Type I error of about or below 10% and a Type II error of about or below 10% is indicative of a risk of having a child with ASD.

A treatment protocol can be personalized to the subject. For instance, a treatment protocol can be personalized based on the metabolites found to be significantly different in a sample obtained from the subject when compared to a control and identified using the method described herein. Such a personalized treatment protocol can include adjusting in the subject the level of the one or combination of metabolites. The treatment protocol can also include adjusting the levels of one or more metabolite associated with the one or combination of two or more metabolites identified as having a level in the biological sample significantly different from the level of the one or combination of metabolites in the control sample. In some aspects, the treatment protocol comprises supplementation with vitamin B12, folate, or combination thereof before and/or during pregnancy.

Another aspect of the present disclosure encompasses a method for determining increased maternal risk of a female subject bearing a child with ASD. The method comprises obtaining or having obtained a biological sample from the female subject; subjecting the sample to methanol extraction; drying the sample extract; reconstituting the sample extract; and measuring the level of one or a combination of two or more metabolites selected from the metabolites listed in Table 1, Table 9, and Table 10 in the reconstituted sample extract using Ultrahigh Performance Liquid Chromatography-Tandem Mass Spectroscopy (UHPLC-MS/MS). The method further comprises applying each of the measured levels of the metabolites against a control panel of metabolite levels created by measuring metabolite levels of the one or combination of metabolites in control subjects with no history of bearing a child with ASD, wherein the panel is stored on a computer system. The method can further comprising removing protein from the biological sample. When the level of one metabolite is measured, the method further comprises comparing the measured level of the metabolite in the sample to the level of the metabolite in the control panel of metabolite levels using a statistical analysis method selected from the standard Student t-test, the Welch test, the Mann-Whitney U test, the Welch t-test, and combinations thereof; and calculating the false discovery rates (FDR; calculates the p value) and optionally the false positive rate (FPR; calculates the q value) for the metabolite. When the levels of a combination of two or more metabolites are measured, the method further comprises calculating the Type I (FPR; false positive rate) and Type II (FNR; false negative rate) errors for the combination of metabolites using FDA or logistic regression.

The method further comprises indicating that the female subject has an increased risk of bearing a child with ASD. When the level of one metabolite is measured, the level of the metabolite in the biological sample is significantly different from the level of the metabolite in the control panel of metabolite levels if the p-value is less than or about 0.05 and the FDR value is less than or about 0.1. When the levels of a combination of two or more metabolites are measured, the Type I error is about or below 10% and the Type II error is about or below 10%. (Specificity and sensitivity)

Yet another aspect of the present disclosure encompasses a method of determining a personalized treatment protocol for a pregnant subject or a subject contemplating conception and at risk of having a child with ASD. The method comprises measuring in a biological sample obtained from the subject the level of one or combination of two or more metabolites selected from the metabolites listed in Table 1, Table 9, and Table 10 and any combination thereof, identifying one or a combination of metabolites having a level in the biological sample significantly different from the level of the one or combination of metabolites in a control sample, and assigning a personalized medical, behavioral, or nutritional treatment protocol to the subject, wherein a level of the one or combination of metabolites in the biological sample significantly different from the level of the one or combination of metabolites in a control sample is indicative of a risk of having a child with ASD. The biological samples, metabolites, and methods of measuring and identifying metabolites of interest can be as described above.

Another aspect of the present disclosure encompasses a method of monitoring the therapeutic effect of an ASD treatment protocol in a pregnant subject or a subject contemplating conception and at risk of having a child with ASD. The method comprises measuring in a first biological sample obtained from the subject the level of one or a combination of metabolites selected from the metabolites listed in Table 1, Table 9, and Table 10 and any combination thereof, measuring in a second biological sample obtained from the subject the level of the one or combination of metabolites, and comparing the level of the one or combination of metabolites in the first sample and the second sample, wherein maintenance of the level of the one or combination of metabolites or a change of the level of the one or combination of metabolites to a level of the one or combination of metabolites in a control sample is indicative that the treatment protocol is therapeutically effective in the subject. The biological samples, metabolites, and methods of measuring and identifying metabolites of interest are as described in this Section above.

The methods provided herein result in, or are aimed at achieving a detectable improvement in one or more indicators or symptoms of ASD in a child born to a subject at risk of bearing a child with ASD. The one or more indicators or symptoms of ASD include, without limitation, changes in eye tracking, skin conductance and/or EEG measurements in response to visual stimuli, difficulties engaging in and responding to social interaction, verbal and nonverbal communication problems, repetitive behaviors, intellectual disability, difficulties in motor coordination, attention issues, sleep disturbances, and physical health issues such as gastrointestinal disturbances.

Several screening instruments are known in the art for evaluating a subject's social and communicative development and thus can be used as aids in screening for and detecting changes in the severity of impairment in communication skills, social interactions, and restricted, repetitive, and stereotyped patterns of behavior characteristic of autism spectrum disorder. Evaluation can include neurologic and genetic assessment, along with in-depth cognitive and language testing. Additional measures developed specifically for diagnosing and assessing autism include the Autism Diagnosis Interview-Revised (ADI-R), the Autism Diagnostic Observation Schedule (ADOS-G) and the Childhood Autism Rating Scale (CARS).

According to CARS, evaluators rate the subject on a scale from 1 to 4 in each of 15 areas: Relating to People; Imitation; Emotional Response; Body Use; Object Use; Adaptation to Change; Visual Response; Listening Response; Taste, Smell, and Touch Response and Use; Fear; Verbal Communication; Nonverbal Communication; Activity; Level an Consistency of Intellectual Response; and General Impressions. A second edition of CARS, known as the Childhood Autism Rating Scale-2 or CARS-2, was developed by Schopler et al. (Childhood Autism Rating Scale Second edition (CARS2): Manual. The original CARS was developed primarily with individuals with co-morbid intellectual functioning and was criticized for not accurately identifying higher functioning individuals with ASD. CARS-2 retained the original CARS form for use with younger or lower functioning individuals (now renamed the CARS2-ST for “Standard Form”), but also includes a separate rating scale for use with higher functioning individuals (named the CARS2-HF for “High Functioning”) and an unscored information-gathering scale (“Questionnaire for Parents or Caregivers” or CARS2-QPC) that has utility for making CARS2ST and CARS2-HF ratings.

Another symptom-rating instrument useful for assessing changes in symptom severity before, during, or following treatment according to a method provided herein is the Aberrant Behavior Checklist (ABC). The ABC is a symptom rating checklist used to assess and classify problem behaviors of children and adults in a variety of settings. The ABC includes 58 items that resolve onto five subscales: (1) irritability/agitation, (2) lethargy/social withdrawal, (3) stereotypic behavior, (4) hyperactivity/noncompliance, and (5) inappropriate speech.

### II. KITS

One aspect of the present disclosure encompasses a kit for performing any of the methods described above. The kit comprises a container for collecting the biological sample from the subject and solutions and solvents for preparing an extract from a biological sample obtained from the subject. The kit further comprises instructions for (i) preparing the extract, (ii) measuring the level of one or more metabolites selected from the metabolites listed in Table 1, Table 9, and Table 10 using Ultrahigh Performance Liquid Chromatography-Tandem Mass Spectroscopy (UPLC-MS/MS); and (iii) applying the measured metabolite levels against a control panel of metabolite levels created by measuring metabolite levels of the one or combination of metabolites in control subjects with no history of bearing a child with ASD.

As used herein, “kits” refer to a collection of elements including at least one non-standard laboratory reagent for use in the disclosed methods, in appropriate packaging, optionally containing instructions for use. A kit may further include any other components required to practice the methods, such as dry powders, concentrated solutions, or ready-to-use solutions. In some aspects, a kit comprises one or more containers that contain reagents for use in the methods. Containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

A kit may include instructions for testing a biological sample of a subject at risk of having a child with ASD. The instructions will generally include information about the use of the kit in the disclosed methods. In other aspects, the instructions may include at least one of the following: description of possible therapies including therapeutic agents; clinical studies; and/or references. The instructions may be printed directly on the container (when present), or as a label applied to the container, or as a separate sheet, pamphlet, card, or folder supplied in or with the container.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “subject” refers to any mammal, including a human, non-human primate, dog, rat, mouse, or guinea pig which suffers, is suspected of or is at risk of having a child with ASD, whether occurring naturally or induced for experimental purposes. In some aspects, the subject is a female subject. In one alternative of the aspects, the subject is a human female subject.

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of autism spectrum disorder, “treat” and “treating” encompass alleviating, ameliorating, delaying the onset of, inhibiting the progression of, or reducing the severity of one or more symptoms associated with an autism spectrum disorder.

As used herein, “therapeutically effective amount” or “pharmaceutically active dose” refers to an amount of a composition which is effective in treating the named disease, disorder or condition.

The terms “sensitivity” and “specificity” are statistical measures of the performance of a binary classification test. Sensitivity (also called the true positive rate, the recall, or probability of detection in some fields) measures the proportion of actual positives that are correctly identified as such (e.g., the percentage of sick people who are correctly identified as having the condition). Specificity (also called the true negative rate) measures the proportion of actual negatives that are correctly identified as such (e.g., the percentage of healthy people who are correctly identified as not having the condition). The terms “positive” and “negative” do not refer to the value of the condition of interest, but to its presence or absence. The condition itself could be a disease, so that “positive” might mean “diseased,” while “negative” might mean “healthy”. In many tests, including diagnostic medical tests, sensitivity is the extent to which actual positives are not overlooked (so false negatives are few), and specificity is the extent to which actual negatives are classified as such (so false positives are few). As such, a highly sensitive test rarely overlooks an actual positive (for example, overlooking a disease condition); a highly specific test rarely registers a positive classification for anything that is not the target of testing (for example, diagnosing a disease condition in a healthy subject); and a test that is highly sensitive and highly specific does both.

A metabolite is a small molecule intermediate or end product of metabolism. Metabolites have various functions, including fuel, structure, signaling, stimulatory and inhibitory effects on enzymes, catalytic activity of their own (usually as a cofactor to an enzyme), defense, and interactions with other organisms (e.g. pigments, odorants, and pheromones). A primary metabolite is directly involved in normal “growth”, development, and reproduction.

The metabolome refers to the complete set of small-molecule chemicals found within a biological sample. The biological sample can be a cell, a cellular organelle, an organ, a tissue, a tissue extract, a biofluid or an entire organism. The small molecule chemicals found in a given metabolome may include both endogenous metabolites that are naturally produced by an organism (such as amino acids, organic acids, nucleic acids, fatty acids, amines, sugars, vitamins, co-factors, pigments, antibiotics, etc.) as well as exogenous chemicals (such as drugs, environmental contaminants, food additives, toxins and other xenobiotics) that are not naturally produced by an organism.

As various changes could be made in the above-described metabolites and methods without departing from the scope of the invention, it is intended that all matter contained in the above description and in the examples given below shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed above are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1: Identification and Characterization of Metabolites Associated with Maternal ASD

Blood samples were collected from 30 mothers of young children with ASD. Control blood samples were also collected from 30 mothers of young typically developing (TD) children. The levels of 55 metabolites measured in the whole blood samples were significantly different (q<0.05) between the 30 mothers of young children with ASD and the 30 mothers of young typically developing (TD) children, after using False Discovery Methods to eliminate false positives. Another 8 metabolites were significantly different for q<0.10. All combinations of 2, 3, 4, and 5 of those metabolites were analyzed to identify the combinations with the highest sensitivity and specificity. Many combinations had positive results. The most significant results were:
Combination of 2 metabolites: N-acetylasparagine and X-12680: sensitivity 83%, specificity 87%.
Combination of 3 metabolites: 6-hydroxyindole sulfate, histidylglutamate, and N-acetylasparagine: sensitivity 90%, specificity 90%.
Combination of 4 metabolites: 6-hydroxyindole sulfate, histidylglutamate, N-acetylasparagine, and N6-carboxymethyllysine: sensitivity 93%, specificity 93%.
Combination of 5 metabolites: 6-hydroxyindole sulfate; histidylglutamate; N-acetylasparagine; N6-carboxymethyllysine; 5alpha-pregnan-3beta, 20alpha-diol disulfate: sensitivity 97%, specificity 93%.

More detailed results are included in Examples 2 and 3 below. Methods used are as detailed in Examples 4 and 5.

Leave-one-out cross-validation was used to determine the best combination to ensure that the results are not just fitted well, but that they are statistically independent. In short, cross-validation leaves out a sample, then determines the best combination of the remaining samples, and finally tests this best combination on the sample that was left out. After this, the sample is put back into the dataset and a different sample is removed whereupon the entire procedure is repeated. This process continues until each sample has been left out one time. This cross-validation procedure ensures that we choose the best combination not just from fitting the results, but from predicting results for the samples which were left out.

Larger sample sizes may result in slightly different combinations of the metabolites being the most significant. The bottom line is that a small set of 2-5 metabolites can be used to differentiate between mothers of young children with ASD and mothers of young TD children, with a high sensitivity and specificity.

These results are the first metabolomics-based measurements of mothers of children with ASD. These results may apply to mothers of younger children, possibly even close to the age of birth, with a somewhat different combination being best for different ages. These metabolites may be different during pregnancy, and possibly even pre-conception, allowing even earlier detection of mothers at high risk of having a child with ASD. However, different reference ranges may need to be established during pregnancy (and different stages of pregnancy), and possibly preconception.

These metabolites may also be useful to monitor the effectiveness of maternal treatment interventions during preconception/pregnancy.

### Example 2: Search for the Most Significant Individual Metabolites

The measurements of the levels of individual metabolites were evaluated using a rich statistical approach. An approach to use for each metabolite was determined. Univariate analysis was performed using hypothesis testing to test for differences between the population mean or median of each group of mothers. Individual metabolite measurements for each group were tested for normality using the Anderson-Darling Test. If both groups accepted the null hypothesis of this test, the F-test was performed to determine if the population variances of each group were equal, which resulted in either the Student's t-test (for equal) or Welch's test (for unequal) being used to test for significant differences between the population means. If at least one of the groups rejected the null hypothesis of the Anderson-Darling test, the two-sample Kolmogorov-Smirnov test was used to determine if the measurements from both groups came from distributions of the same shape. If the samples accepted the null hypothesis of the Kolmogorov-Smirnov test, the Mann-Whitney U test was used to test for significant differences between the medians of the two samples. If the samples rejected the null hypothesis of the Kolmogorov-Smirnov test, the Welch's test should be used to test for significant differences in the population mean. Each test was done with a significance of 5%. Then, False Discovery Rate (FDR) methods were used to correct for multiple-hypothesis testing. This resulted in a set of 63 metabolites that had p<0.05 and FDR<0.1. See Table 1.

TABLE 1
Most Significant Metabolites.

| | | | | | | ASD/Control |
|---|---|---|---|---|---|---|
| Measurements | Super Pathway | SubPathway | Test | p-Value | FDR | mean |
| histidylglutamate | Peptide | Dipeptide | ‘t!=’ | 3.52E−05 | 0.00E+00 | 1.44 |
| decanoylcarnitine (C10) | Lipid | Fatty Acid | ‘t!=’ | 7.78E−05 | 0.00E+00 | 0.63 |
| | | Metabolism(Acyl | | | | |
| | | Carnitine) | | | | |
| X - 12459 | | | ‘mannW’ | 0.000130115 | 0 | 0.56 |
| octanoylcarnitine (C8) | Lipid | Fatty Acid | ‘mannW’ | 0.000253058 | 0 | 0.65 |
| | | Metabolism(Acyl | | | | |
| | | Carnitine) | | | | |
| cis-4-decenoylcarnitine | Lipid | Fatty Acid | ‘t=’ | 0.000376168 | 0 | 0.72 |
| (C10:1) | | Metabolism(Acyl | | | | |
| | | Carnitine) | | | | |
| fructose | Carbohydrate | Fructose, Mannose | ‘t!=*’ | 0.000455891 | 0 | 0.58 |
| | | and Galactose | | | | |
| | | Metabolism | | | | |
| X - 12680 | | | ‘mannW’ | 0.000534659 | 0 | 0.43 |
| S-1-pyrroline-5- | Amino Acid | Glutamate | ‘mannW’ | 0.0008331 | 0 | 0.63 |
| carboxylate | | Metabolism | | | | |
| N-palmitoylglycine | Lipid | Fatty Acid | ‘t=’ | 0.001183685 | 0 | 0.77 |
| | | Metabolism(Acyl | | | | |
| | | Glycine) | | | | |
| gamma-glutamylglycine | Peptide | Gamma-glutamyl | ‘t!=*’ | 0.001187593 | 0 | 0.25 |
| | | Amino Acid | | | | |
| X - 13729 | | | ‘mannW’ | 0.001235408 | 0 | 0.61 |
| N-acetylasparagine | Amino Acid | Alanine and | ‘mannW’ | 0.001370333 | 0 | 0.74 |
| | | Aspartate | | | | |
| | | Metabolism | | | | |
| 4-vinylphenol sulfate | Xenobiotics | Benzoate | ‘t!=*’ | 0.001380519 | 0 | 0.30 |
| | | Metabolism | | | | |
| 6-hydroxyindole sulfate | Xenobiotics | Chemical | ‘t!=’ | 0.001685958 | 0 | 0.58 |
| N-formylanthranilic acid | Amino Acid | Tryptophan | ‘t=’ | 0.001996329 | 0 | 0.68 |
| | | Metabolism | | | | |
| N-acetyl-2-aminooctanoate* | Lipid | Fatty Acid, Amino | ‘t!=’ | 0.002200654 | 0 | 0.52 |
| X - 23639 | | | ‘mannW’ | 0.002499392 | 0 | 0.74 |
| laurylcarnitine (C12) | Lipid | Fatty Acid | ‘mannW’ | 0.003033948 | 0 | 0.72 |
| | | Metabolism(Acyl | | | | |
| | | Carnitine) | | | | |
| asparaginylalanine | Peptide | Dipeptide | ‘t=’ | 0.003548999 | 0 | 1.27 |
| 3-indoxyl sulfate | Amino Acid | Tryptophan | ‘t!=’ | 0.003805197 | 0 | 0.66 |
| | | Metabolism | | | | |
| citrulline | Amino Acid | Urea cycle; Arginine | ‘t=’ | 0.003893808 | 0 | 0.88 |
| | | and Proline | | | | |
| | | Metabolism | | | | |
| X - 21310 | | | ‘t!=’ | 0.003941848 | 0 | 0.67 |
| arachidoylcarnitine | Lipid | Fatty Acid | ‘mannW’ | 0.004032978 | 0 | 0.83 |
| (C20)* | | Metabolism(Acyl | | | | |
| | | Carnitine) | | | | |
| glycine | Amino Acid | Glycine, Serine and | ‘t=’ | 0.004196826 | 0 | 0.83 |
| | | Threonine | | | | |
| | | Metabolism | | | | |
| 5-oxoproline | Amino Acid | Glutathione | ‘t=’ | 0.004462643 | 0 | 0.91 |
| | | Metabolism | | | | |
| X - 12411 | | | ‘t!=*’ | 0.004705641 | 0 | 0.48 |
| X - 24106 | | | ‘t=’ | 0.006125122 | 0 | 0.87 |
| 7-methylxanthine | Xenobiotics | Xanthine | ‘mannW’ | 0.006540099 | 0 | 0.57 |
| | | Metabolism | | | | |
| myristoylcarnitine | Lipid | Fatty Acid | ‘mannW’ | 0.006668876 | 0 | 0.80 |
| (C14) | | Metabolism(Acyl | | | | |
| | | Carnitine) | | | | |
| catechol sulfate | Xenobiotics | Benzoate | ‘mannW’ | 0.00728836 | 0 | 0.65 |
| | | Metabolism | | | | |
| N-palmitoylserine | Lipid | Endocannabinoid | ‘mannW’ | 0.007570062 | 0 | 0.75 |
| phenol sulfate | Amino Acid | Tyrosine Metabolism | ‘mannW’ | 0.008684371 | 0 | 0.65 |
| propionylglycine | Lipid | Fatty Acid | ‘t!=*’ | 0.008747685 | 0 | 0.45 |
| | | Metabolism (also | | | | |
| | | BCAA Metabolism) | | | | |
| isovalerylglycine | Amino Acid | Leucine, Isoleucine | ‘mannW’ | 0.010086564 | 0 | 0.76 |
| | | and Valine | | | | |
| | | Metabolism | | | | |
| S-methylglutathione | Amino Acid | Glutathione | ‘t=’ | 0.010418864 | 0 | 0.82 |
| | | Metabolism | | | | |
| gamma-glutamyltyrosine | Peptide | Gamma-glutamyl | ‘mannW’ | 0.010810343 | 0 | 0.62 |
| | | Amino Acid | | | | |
| stearoylcarnitine (C18) | Lipid | Fatty Acid | ‘mannW’ | 0.011227764 | 0 | 0.84 |
| | | Metabolism(Acyl | | | | |
| | | Carnitine) | | | | |
| lignoceroylcarnitine | Lipid | Fatty Acid | ‘t=’ | 0.012277565 | 0 | 0.80 |
| (C24)* | | Metabolism(Acyl | | | | |
| | | Carnitine) | | | | |
| X - 15220 | | | ‘t!=’ | 0.012440634 | 0 | 1.43 |
| X - 21286 | | | ‘mannW’ | 0.012662745 | 0 | 0.70 |
| glutamine | Amino Acid | Glutamate | ‘t=’ | 0.014704699 | 0 | 0.92 |
| | | Metabolism | | | | |
| alpha-ketoglutaramate* | Amino Acid | Glutamate | ‘t=’ | 0.01488295 | 0 | 0.71 |
| | | Metabolism | | | | |
| cinnamoylglycine | Xenobiotics | Food | ‘t!=*’ | 0.015627063 | 0 | 0.46 |
| | | Component/Plant | | | | |
| X - 15461 | | | ‘t=’ | 0.016214421 | 0 | 0.81 |
| docosapentaenoylcarnitine | Lipid | Fatty Acid | ‘mannW’ | 0.017642728 | 0 | 0.71 |
| (C22:5n3)* | | Metabolism(Acyl | | | | |
| | | Carnitine) | | | | |
| proline | Amino Acid | Urea cycle; Arginine | ‘mannW’ | 0.019112397 | 0 | 0.87 |
| | | and Proline | | | | |
| | | Metabolism | | | | |
| succinylcarnitine (C4-DC) | Energy | TCA Cycle | ‘t=’ | 0.025397966 | 0 | 0.85 |
| N-acetylvaline | Amino Acid | Leucine, Isoleucine | ‘t=’ | 0.035567956 | 0 | 0.77 |
| | | and Valine | | | | |
| | | Metabolism | | | | |
| X - 18886 | | | ‘mannW’ | 0.022343738 | 0.004915 | 1.92 |
| N-acetylleucine | Amino Acid | Leucine, Isoleucine | ‘mannW’ | 0.020685552 | 0.005461 | 0.51 |
| | | and Valine | | | | |
| | | Metabolism | | | | |
| guaiacol sulfate | Xenobiotics | Benzoate | ‘mannW’ | 0.016954881 | 0.0071 | 0.78 |
| | | Metabolism | | | | |
| X - 24813 | | | ‘t=’ | 0.009131972 | 0.008192 | 0.80 |
| X - 12216 | | | ‘mannW’ | 0.009879299 | 0.0284 | 0.76 |
| N-acetylhistidine | Amino Acid | Histidine Metabolism | ‘mannW’ | 0.023921165 | 0.030038 | 0.72 |
| 3-methylxanthine | Xenobiotics | Xanthine | ‘mannW’ | 0.025030586 | 0.042054 | 0.73 |
| | | Metabolism | | | | |
| iminodiacetate (IDA) | Xenobiotics | Chemical | ‘mannW’ | 0.024156885 | 0.057346 | 1.24 |
| malonylcarnitine | Lipid | Fatty Acid Synthesis | ‘t!=’ | 0.069313243 | 0.061169 | 0.82 |
| 5-dodecenoylcarnitine | Lipid | Fatty Acid | ‘mannW’ | 0.026077477 | 0.063353 | 0.78 |
| (C12:1) | | Metabolism(Acyl | | | | |
| | | Carnitine) | | | | |
| N-acetylglycine | Amino Acid | Glycine, Serine and | ‘mannW’ | 0.024156885 | 0.067176 | 0.74 |
| | | Threonine | | | | |
| | | Metabolism | | | | |
| X - 15503 | | | ‘mannW’ | 0.010762613 | 0.070453 | 0.94 |
| nicotinamide adenine | Cofactors and | Nicotinate and | ‘t!=*’ | 0.033290517 | 0.072638 | 0.41 |
| dinucleotide (NAD+) | Vitamins | Nicotinamide | | | | |
| | | Metabolism | | | | |
| 5-methylthioadenosine | Amino Acid | Polyamine | ‘mannW’ | 0.025101283 | 0.092299 | 0.84 |
| (MTA) | | Metabolism | | | | |
| arachidonoylcarnitine | Lipid | Fatty Acid | ‘mannW’ | 0.025101283 | 0.098307 | 0.76 |
| (C20:4) | | Metabolism(Acyl | | | | |
| | | Carnitine) | | | | |

Test refers to the type of statistical test that was used, namely “t=” refers to the Student's t-test, “t!=” refers to the Welch's test, “t!=*” refers to the Welch's test without the normality criteria being met, and “mannW” refers to the Mann-Whitney U test.

### Example 3: Search for Combinations of Metabolites to Best Differentiate the Two Groups of Mothers

FDA methods were used to search for combinations of metabolites that best differentiated the two groups of mothers. An exhaustive search was performed with the 63 most significant metabolites, using combinations of 1, 2, 3, 4, and 5 metabolites. In the tables below, the 15 best individual metabolites are listed, followed by the 15 best combinations of two metabolites, followed by the 15 best combinations of three metabolites, followed by the 15 best combinations of four metabolites, and finally the 15 best combinations of five metabolites. “Best” was defined by the metabolites being able to predict if a mother belonged to the high-risk (i.e., already had a child diagnosed with ASD) or regular risk group (has not had a child diagnosed with ASD). In other words, sensitivity and specificity were computed, and the best metabolites were those that maximized the sensitivity and specificity.

The top 15 combinations for different numbers of metabolites are shown in Tables 2-6 below. The most promising candidates are the following three combinations of three metabolites, as they each resulted in misclassification errors of 10%: (1) the combination of 6-hydroxyindole sulfate; histidylglutamate; N-acetylasparagine; (2) the combination of histidylglutamate; N-acetylasparagine; X-21310; and (3) the combination of 3-indoxyl sulfate; histidylglutamate; N-acetylasparagine.

Also, by looking at all the best combinations for all numbers of metabolites, there are some metabolites which appeared several times:
Histidylglutamate, which according to HMDB, is a dipeptide composed of histidine and glutamate. It is an incomplete breakdown product of protein digestion or protein catabolism.
N-acetylasparagine, which according to HMDB, is produced by the degradation of asparagine.

TABLE 2
Top 1

| | Type I | Type II |
|---|---|---|
| Metabolites | Error | Error |
| decanoylcarnitine (C10) | 33.333% | 23.333% |
| X - 12459 | 26.667% | 23.333% |
| Fructose | 30.000% | 26.667% |
| 7-methylxanthine | 36.667% | 33.333% |
| Octanoylcarnitine (C8) | 33.333% | 23.333% |
| Cis-4-decenoylcarnitine (C10:1) | 33.333% | 30.000% |
| X - 12680 | 30.000% | 33.333% |
| Nicotinamide adenine dinucleotide (NAD+) | 40.000% | 36.667% |
| 4-vinylphenol sulfate | 33.333% | 30.000% |
| Gamma-glutamylglycine | 40.000% | 23.333% |
| 6-hydroxyindole sulfate | 36.667% | 30.000% |
| N-acetyl-2-aminooctanoate* | 36.667% | 40.000% |
| S-1-pyrroline-5-carboxylate | 30.000% | 26.667% |
| N-acetylasparagine | 33.333% | 30.000% |
| X - 12411 | 36.667% | 40.000% |

TABLE 3
Top 2

| | Type I | Type II |
|---|---|---|
| Metabolites | Error | Error |
| N-acetylasparagine; X - 12680 | 16.667% | 13.333% |
| 3-indoxyl sulfate; histidylglutamate | 16.667% | 16.667% |
| Histidylglutamate; X - 21310 | 16.667% | 16.667% |
| Laurylcarnitine (C12); S-1-pyrroline-5- | 30.000% | 30.000% |
| carboxylate | | |
| 6-hydroxyindole sulfate; histidylglutamate | 16.667% | 13.333% |
| Decanoylcarnitine (C10); fructose | 23.333% | 23.333% |
| N-acetylasparagine; X - 21310 | 23.333% | 20.000% |
| Histidylglutamate; X - 24813 | 20.000% | 20.000% |
| Gamma-glutamylglycine; X - 12459 | 20.000% | 23.333% |
| 4-vinylphenol sulfate; N-acetyl-2- | 20.000% | 16.667% |
| aminooctanoate* | | |
| 5-oxoproline; phenol sulfate | 23.333% | 23.333% |
| 4-vinylphenol sulfate; fructose | 23.333% | 23.333% |
| Fructose; S-1-pyrroline-5-carboxylate | 23.333% | 26.667% |
| Fructose; glutamine | 26.667% | 23.333% |
| Fructose; histidylglutamate | 13.333% | 23.333% |

TABLE 4
Top 3

| | Type I | Type II |
|---|---|---|
| Metabolites | Error | Error |
| 6-hydroxyindole sulfate; histidylglutamate; N- | 10.000% | 10.000% |
| acetylasparagine | | |
| Fructose; histidylglutamate; X - 21310 | 20.000% | 10.000% |
| histidylglutamate; N-acetylasparagine; X - 21310 | 10.000% | 10.000% |
| 3-indoxyl sulfate; histidylglutamate; | 10.000% | 10.000% |
| N-acetylasparagine | | |
| 6-hydroxyindole sulfate; histidylglutamate; S- | 16.667% | 10.000% |
| methylglutathione | | |
| 6-hydroxyindole sulfate; fructose; histidylgluta- | 16.667% | 10.000% |
| mate | | |
| Histidylglutamate; N-acetylasparagine; N- | 16.667% | 13.333% |
| formylanthranilic acid | | |
| 6-hydroxyindole sulfate; arachidonoylcarnitine | 26.667% | 10.000% |
| (C20:4); histidylglutamate | | |
| 3-indoxyl sulfate; fructose; histidylgluta- | 20.000% | 10.000% |
| mate | | |
| 6-hydroxyindole sulfate; docosapentaenoylcarni- | 23.333% | 13.333% |
| tine (C22:5n3)*; histidylglutamate | | |
| Docosapentaenoylcarnitine (C22:5n3)*; | 30.000% | 6.667% |
| histidylglutamate; X - 12680 | | |
| 3-indoxyl sulfate; histidylglutamate; S- | 16.667% | 10.000% |
| methylglutathione | | |
| Histidylglutamate; N-acetylasparagine; X - 12680 | 16.667% | 6.667% |
| N-acetyl-2-aminooctanoate*; octanoylcarnitine | 26.667% | 10.000% |
| (C8); X - 15220 | | |
| Histidylglutamate; N-formylanthranilic acid; | 16.667% | 10.000% |
| X - 15461 | | |

TABLE 5
Top 4

| | Type I | Type II |
|---|---|---|
| Metabolites | Error | Error |
| Glutamine; histidylglutamate; N-formylanthranilic | 16.667% | 10.000% |
| acid; X - 15461 | | |
| 4-vinylphenol sulfate; histidylglutamate; N- | 10.000% | 10.000% |
| formylanthranilic acid; X - 15461 | | |
| Cinnamoylglycine; decanoylcarnitine (C10); | 13.333% | 10.000% |
| X - 15220; X - 24813 | | |
| Cis-4-decenoylcarnitine (C10:1); histidylgluta- | 13.333% | 6.667% |
| mate; X - 15220; X - 24813 | | |
| 6-hydroxyindole sulfate; histidylglutamate; N- | 10.000% | 10.000% |
| acetylasparagine; X - 15461 | | |
| 3-indoxyl sulfate; histidylglutamate; N- | 10.000% | 13.333% |
| acetylasparagine; X - 15461 | | |
| Histidylglutamate; N-acetylasparagine; X - | 10.000% | 10.000% |
| 15461; X - 21310 | | |
| Histidylglutamate; N-acetylasparagine; X - | 10.000% | 10.000% |
| 12680; X - 15461 | | |
| Alpha-ketoglutaramate*; decanoylcarnitine | 23.333% | 6.667% |
| (C10); histidylglutamate; X - 15461 | | |
| Histidylglutamate; N-acetylasparagine; N- | 13.333% | 13.333% |
| acetylvaline; X - 21310 | | |
| 3-indoxyl sulfate; fructose; histidylglutamate; | 20.000% | 10.000% |
| X - 15461 | | |
| histidylglutamate; N-acetylasparagine; S- | 10.000% | 10.000% |
| methylglutathione; X - 21310 | | |
| Histidylglutamate; N-palmitoylglycine; X - | 13.333% | 13.333% |
| 15461; X - 21310 | | |
| 6-hydroxyindole sulfate; histidylglutamate; | 6.667% | 10.000% |
| N-acetylasparagine; S-methylglutathione | | |
| Histidylglutamate; N-formylanthranilic acid; | 10.000% | 10.000% |
| N-palmitoylglycine; X - 15461 | | |

TABLE 6
Top 5

| | Type I | Type II |
|---|---|---|
| Metabolites | Error | Error |
| Alpha-ketoglutaramate*; histidylglutamate; N- | 13.333% | 6.667% |
| formylanthranilic acid; N-palmitoylglycine; | | |
| X - 15461 | | |
| Gamma-glutamylglycine; histidylglutamate; N- | 10.000% | 10.000% |
| acetylasparagine; N-formylanthranilic acid; | | |
| X - 15461 | | |
| Glycine; histidylglutamate; N-acetylasparagine; | 6.667% | 10.000% |
| N-formylanthranilic acid; X - 15461 | | |
| Cinnamoylglycine; histidylglutamate; N- | 6.667% | 10.000% |
| acetylasparagine; X - 12680; X - 15461 | | |
| Glutamine; histidylglutamate; N-acetylasparagine; | 3.333% | 10.000% |
| N-formylanthranilic acid; X - 15461 | | |
| Cinnamoylglycine; histidylglutamate; laurylcarni- | 10.000% | 10.000% |
| tine (C12); X - 12680; X - 15461 | | |
| Histidylglutamate; N-acetylasparagine; N- | 3.333% | 10.000% |
| formylanthranilic acid; proline; X - 15461 | | |
| Gamma-glutamylturosine; histidylglutamate; N- | 3.333% | 10.000% |
| acetylasparagine; N-formylanthranilic acid; | | |
| X - 15461 | | |
| Histidylglutamate; N-acetylasparagine; N- | 3.333% | 6.667% |
| formylanthranilic acid; nicotinamide adenine | | |
| dinucleotide (NAD+); X - 15461 | | |
| Histidylglutamate; N-acetylasparagine; N- | 3.333% | 10.000% |
| palmitoylglycine; X - 15461; X - 21310 | | |
| Histidylglutamate; N-acetylasparagine; X - 12680; | 3.333% | 10.000% |
| X - 15461; X - 21310 | | |
| 6-hydroxyindole sulfate; histidylglutamate; N- | 6.667% | 16.667% |
| acetylasparagine; X - 15461; X - 15503 | | |
| 3-indoxyl sulfate; histidylglutamate; N- | 10.000% | 13.333% |
| acetylasparagine; X - 15461; X - 15503 | | |
| Histidylglutamate; N-acetylasparagine; X- 12411; | 10.000% | 13.333% |
| X - 15461; X - 21310 | | |
| Glutamine; histidylglutamate; N-acetylasparagine; | 13.333% | 13.333% |
| X - 15461; X - 21310 | | |

### Example 4: Sample Collection

Fasting whole blood samples were collected from mothers in the morning. Fasting was important to reduce random fluctuations due to diet. Morning collection was used to increase uniformity. Whole blood was used to be able to capture metabolites that exist primarily inside of cells as well as those that primarily exist outside of cells, allowing a more comprehensive understanding of metabolism. This is important because most studies focus only on serum or plasma, and hence miss metabolites that existi primarily inside cells.

After collection, samples were frozen in a −80° C. freezer. Once all samples were collected from all patients, they were sent together to Metabolon on dry ice. It is important to test them all together in one batch because the test is semi-quantitative, i.e., the test measures relative, not absolute, differences between the samples. Also, all the samples were collected during the same time period so the difference in storage times between the two groups was small, which also helps to minimize differences since even at −80° C. there is a small degradation of sample quality (estimated at 2%/year).

It is important to note that the two participant groups were closely matched in age (34.9±5.2 years and 34.7±5.7 years for the mothers of ASD children and typically-developing children, respectively), and all had children ages 2-5 years old. Since autism is diagnosed at an average age of 4.5 years in Arizona, that is about as close to birth as can be easily obtained from a study in which you select mothers of children who are diagnosed with autism. So the close matching in age of the mothers, and the relatively close time to when they gave birth, helped eliminate random fluctuations due to age, and is a reasonable estimate of their status during pregnancy/nursing, when their metabolic status is most likely to influence their infant's development of autism.

### Example 5: Methodology of Measuring Metabolites Using the Metabolon System

Sample Acquisition. Following receipt, samples were inventoried and immediately stored at −80° C. Each sample received was accessioned into the Metabolon LIMS system and was assigned by the LIMS a unique identifier that was associated with the original source identifier only. This identifier was used to track all sample handling, tasks, results, etc. The samples (and all derived aliquots) were tracked by the LIMS system. All portions of any sample were automatically assigned their own unique identifiers by the LIMS when a new task was created; the relationship of these samples was also tracked. All samples were maintained at −80° C. until processed.

Sample Preparation: Samples were prepared using the automated MicroLab STAR® system from Hamilton Company. Several recovery standards were added prior to the first step in the extraction process for QC purposes. To remove protein, dissociate small molecules bound to protein or trapped in the precipitated protein matrix, and to recover chemically diverse metabolites, proteins were precipitated with methanol under vigorous shaking for 2 min (Glen Mills GenoGrinder 2000) followed by centrifugation. The resulting extract was divided into five fractions: two for analysis by two separate reverse phase (RP)/UPLC-MS/MS methods with positive ion mode electrospray ionization (ESI); one for analysis by RP/UPLC-MS/MS with negative ion mode ESI; one for analysis by HILIC/UPLC-MS/MS with negative ion mode ESI; and one sample was reserved for backup. Samples were placed briefly on a TurboVap® (Zymark) to remove the organic solvent. The sample extracts were stored overnight under nitrogen before preparation for analysis.

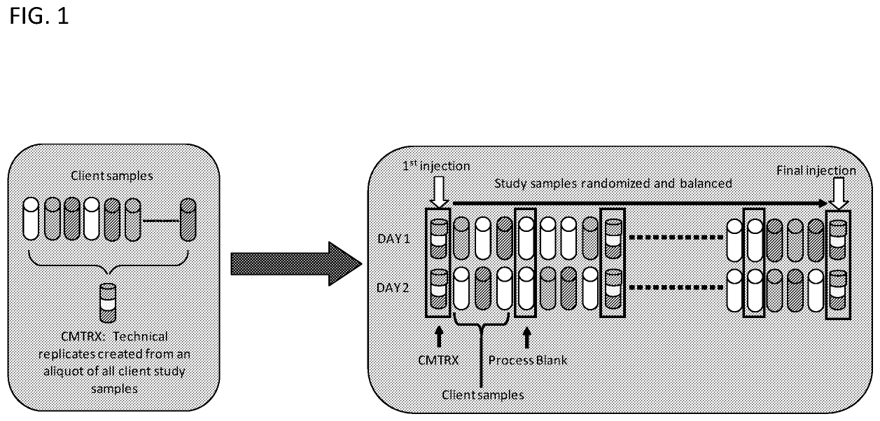

QA/QC: Several types of controls were analyzed in concert with the experimental samples: a pooled matrix sample generated by taking a small volume of each experimental sample (or alternatively, use of a pool of well-characterized human plasma) served as a technical replicate throughout the data set; extracted water samples served as process blanks; and a cocktail of QC standards that were carefully chosen not to interfere with the measurement of endogenous compounds were spiked into every analyzed sample, allowed instrument performance monitoring, and aided chromatographic alignment. Tables 7 and 8 describe these QC samples and standards. Instrument variability was determined by calculating the median relative standard deviation (RSD) for the standards that were added to each sample prior to injection into the mass spectrometers. Overall process variability was determined by calculating the median RSD for all endogenous metabolites (i.e., non-instrument standards) present in 100% of the pooled matrix samples. Experimental samples were randomized across the platform run with QC samples spaced evenly among the injections, as outlined in FIG. 1.

TABLE 7
Description of Metabolon QC Samples

| Type | Description | Purpose |
|---|---|---|
| MTRX | Large pool of human | Assure that all aspects of the |
| | plasma maintained by | Metabolon process are operating |
| | Metabolon that has been | within specifications. |
| | characterized | |
| | extensively. | |
| CMTRX | Pool created by taking | Assess the effect of a non-plasma |
| | a small aliquot from | matrix on the Metabolon process |
| | every customer sample. | and distinguish biological |
| | | variability from process |
| | | variability. |
| PRCS | Aliquot of ultra-pure | Process Blank used to assess the |
| | water | contribution to compound signals |
| | | from the process. |
| SOLV | Aliquot of solvents used | Solvent Blank used to segregate |
| | in extraction. | contamination sources in the |
| | | extraction. |

TABLE 8
Metabolon QC Standards

| Type | Description | Purpose |
|---|---|---|
| RS | Recovery Standard | Assess variability and verify performance |
| | | of extraction and instrumentation. |
| IS | Internal Standard | Assess variability and performance of |
| | | instrument. |

Ultrahigh Performance Liquid Chromatography-Tandem Mass Spectroscopy (UPLC-MS/MS): All methods utilized a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in solvents compatible to each of the four methods. Each reconstitution solvent contained a series of standards at fixed concentrations to ensure injection and chromatographic consistency. One aliquot was analyzed using acidic positive ion conditions, chromatographically optimized for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot was also analyzed using acidic positive ion conditions; however it was chromatographically optimized for more hydrophobic compounds. In this method, the extract was gradient eluted from the same aforementioned C18 column using methanol, acetonitrile, water, 0.05% PFPA and 0.01% FA, and was operated at an overall higher organic content. Another aliquot was analyzed using basic negative ion optimized conditions using a separate dedicated C18 column. The basic extracts were gradient eluted from the column using methanol and water, however with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternated between MS and data-dependent MSn scans using dynamic exclusion. The scan range varied slightly between methods but covered 70-1000 m/z. Raw data files were archived and extracted as described below.

Bioinformatics: The informatics system consisted of four major components, the Laboratory Information Management System (LIMS), the data extraction and peak-identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition.

LIMS: The purpose of the Metabolon LIMS system was to enable fully auditable laboratory automation through a secure, easy to use, and highly specialized system. The scope of the Metabolon LIMS system encompasses sample accessioning, sample preparation and instrumental analysis, and reporting and advanced data analysis. All of the subsequent software systems are grounded in the LIMS data structures. It has been modified to leverage and interface with the in-house information extraction and data visualization systems, as well as third party instrumentation and data analysis software.

Data Extraction and Compound Identification: Raw data was extracted, peak-identified and QC processed using Metabolon's hardware and software. These systems are built on a web-service platform utilizing Microsoft's.NET technologies, which run on high-performance application servers and fiber-channel storage arrays in clusters to provide active failover and load-balancing. Compounds were identified by comparison to library entries of purified standards or recurrent unknown entities. Metabolon maintains a library based on authenticated standards that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library+/−10 ppm, and the MS/MS forward and reverse scores between the experimental data and authentic standards. The MS/MS scores are based on a comparison of the ions present in the experimental spectrum to the ions present in the library spectrum. While there may be similarities between these molecules based on one of these factors, the use of all three data points can be utilized to distinguish and differentiate biochemicals. More than 3300 commercially available purified standard compounds have been acquired and registered into LIMS for analysis on all platforms for determination of their analytical characteristics. Additional mass spectral entries have been created for structurally unnamed biochemicals, which have been identified by virtue of their recurrent nature (both chromatographic and mass spectral). These compounds have the potential to be identified by future acquisition of a matching purified standard or by classical structural analysis.

Curation: A variety of curation procedures were carried out to ensure that a high quality data set was made available for statistical analysis and data interpretation. The QC and curation processes were designed to ensure accurate and consistent identification of true chemical entities, and to remove those representing system artifacts, mis-assignments, and background noise. Metabolon data analysts use proprietary visualization and interpretation software to confirm the consistency of peak identification among the various samples. Library matches for each compound were checked for each sample and corrected if necessary.

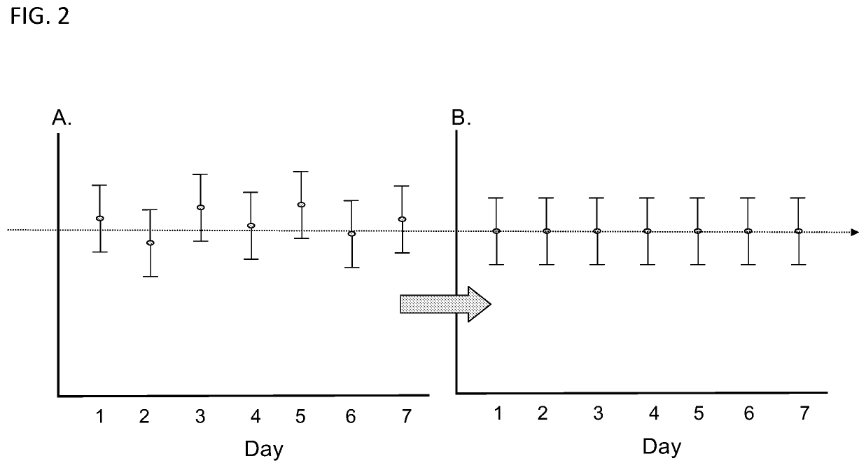

Metabolite Quantification and Data Normalization: Peaks were quantified using area-under-the-curve. For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”; FIG. 2). For studies that did not require more than one day of analysis, no normalization is necessary, other than for purposes of data visualization. In certain instances, biochemical data may have been normalized to an additional factor (e.g., cell counts, total protein as determined by Bradford assay, osmolality, etc.) to account for differences in metabolite levels due to differences in the amount of material present in each sample.

### Example 6. Determining the Risk of Having a Child with ASD for a Pregnant Woman

A whole blood sample is collected from a pregnant woman to determine the risk of having a child with ASD. The level of one metabolite selected from Table 2, and/or the levels of two or more metabolites selected from Tables 3-6 are measured in the blood sample. The level(s) of the measured metabolite(s) is compared to the level(s) of the biomarker(s) in a control sample obtained from mothers of typically developing children. The level(s) of the measured metabolite(s) is found to be different from the level(s) of metabolite(s) in the control sample, and the woman is informed that she is at risk of having a child with ASD with a high level of certainty.

### Example 7. Determining the Risk of Having a Child with ASD for a Woman Contemplating Pregnancy

A whole blood sample is collected from a woman contemplating pregnancy to determine the risk of having a child with ASD. The level of one metabolite selected from Table 2, and/or the levels of two or more metabolites selected from Tables 3-6 are measured in the blood sample. The level(s) of the measured metabolite(s) is compared to the level(s) of the biomarker(s) in a control sample obtained from mothers of typically developing children. The level(s) of the measured metabolite(s) is found to be different from the level(s) of metabolite(s) in the control sample, and the woman is informed that she is at risk of having a child with ASD with a high level of certainty.

### Example 8. Altered Metabolism of Mothers of Young Children with Autism Spectrum Disorder

Autism spectrum disorder (ASD) involves a combination of abnormal social communication, stereotyped behaviors, and restricted interests. ASD is assumed to be caused by complex interactions between genetic and environmental factors, both of which can affect metabolism. Previous studies by the inventors have revealed significant abnormalities in the folate-one carbon metabolism and the transsulfuration pathways of children with ASD and their mothers, resulting in decreased methylation capability, decreased glutathione levels, and increased oxidative stress. Furthermore, the presence of mutations in the MTHFR gene was found to be associated with increased risk of ASD. Additionally, levels of prenatal vitamins taken during pregnancy that include B12 and folate are associated with a decreased ASD risk, suggesting an association of metabolite levels of the folate one-carbon metabolism (FOCM) and the transsulfuration pathway (TS) pathways with ASD. Studies found that maternal gene variants in the one-carbon metabolism pathway were associated with increased ASD risk when there was no or only low levels of periconceptional prenatal vitamin intake.

Additional metabolic differences may also be present in mothers of children with autism, but there has been relatively little investigation of their metabolic state. A more comprehensive understanding of metabolites and metabolic pathways of mothers of children with ASD may lead to a better understanding of the etiology of autism and provide some insights for evaluating pre-conception risk and/or risk during pregnancy. For example, currently, the general risk of having a child with ASD in the US is approximately 1.7%, however, the recurrence risk increases to approximately 19% if the mother already has a child diagnosed with ASD.

In this example, the metabolic profile of mothers of young children with autism and mothers of typically developing children, 2-5 years after birth were analyzed. Measurements were conducted with whole blood to provide information on both intra-cellular and extra-cellular metabolism. This study was limited to women who were not taking folate, B12, or multi-vitamin/mineral supplements during the 2 months prior to sample collection, in order to minimize the effect of supplements on metabolism. The study includes assessments of many different aspects of metabolism, including analysis of amino acids, peptides, carbohydrates, lipids, nucleotides, Kreb's cycle, vitamins/co-factors, and xenobiotics.

### Methodology.

Participants. The inclusion criteria were: 1) Mother of a child 2-5 years of age; 2) Child has ASD or has typical development (TD) including both neurological and physical development; and 3) ASD diagnosis verified by the Autism Diagnostic Interview-Revised (ADI-R).

The exclusion criteria were: 1) Currently taking a vitamin/mineral supplement containing folic acid and/or vitamin B12; and 2) Pregnant or planning to become pregnant in the next six months

Diet. An estimate of dietary intake during the previous week was obtained using Block Brief 2000 Food Frequency Questionnaire (Adult version), from Nutrition Quest (www.nutritionquest.com).

Biological Sample Collection. Fasting whole blood samples were collected in the morning at the Mayo Clinic. Samples were stored at −80° C. freezers at Mayo and ASU until all samples were collected, and then all samples were sent together to Metabolon for testing.

Vitamin B12 (cyanocobalamin) was measured quantitatively with a Beckman Coulter Access competitive binding immunoenzymatic assay. Briefly, serum is treated with alkaline potassium cyanide and dithiothreitol to denature binding proteins and convert all forms of vitamin B12 to cyanocobalamin. Cyanocobalamin from the serum competes against particle-bound anti-intrinsic factor antibody for binding to intrinsic factor—alkaline phosphatase conjugate. After washing, alkaline phosphatase activity on a chemiluminescent substrate is measured and compared against a multi-point calibration curve of known cyanocobalamin concentrations.

Folate (vitamin B9) was measured quantitatively with a Beckman Coulter Access competitive binding receptor assay. Briefly, serum folate competes against a folic acid—alkaline phosphatase conjugate for binding to solid phase-bound folate binding protein. After washing, alkaline phosphatase activity on a chemiluminescent substrate is measured and compared against a multi-point calibration curve of known folate concentrations. The Folate assay is designed to have equal affinities for Pteroylglutamic acid (Folic acid) and 5-Methyltetrahydrofolic acid (Methyl-THF), so the result is a measure of both.

Methylmalonic acid was measured quantitatively by liquid chromatography tandem mass spectrometry (LC-MS/MS). Briefly, serum is mixed with d3-methylmalonic acid as an internal standard, isolated by solid phase extraction, separated on a C18 column, and analyzed in negative ion mode. Chromatographic conditions and mass transitions were chosen to carefully distinguish methylmalonic acid from succinic acid.

Homocysteine was measured quantitatively by LC-MS/MS. Serum is spiked with d8-homocystine as an internal standard, reduced to break disulfide bonds, and deproteinized with formic acid and trifluoroacetic acid in acetonitrile. Measurement of total homocysteine and d4-homocysteine (reduced from d8-homocystine) is performed in positive ion mode with electrospray ionization.

Urine F2-Isoprostane (8-isoprostane) was measured quantitatively by LC-MS/MS after separation from prostaglandin F2 alpha. Urine is spiked with deuterated F2-isoprostane and deuterated prostaglandin F2 alpha, then positive pressure filtered. A mixed mode anion exchange turbulent flow column is used to clean up samples which are then separated on a C8 column and analyzed in negative ion mode.

Vitamin D (25-hydroxyvitamin D2 and D3) was measured quantitatively by LC-MS/MS. D6-25-hydroxyvitamin D3 is added to serum as an internal standard before protein precipitation with acetonitrile. Online turbulent flow chromatography is used to further clean up the samples prior to separation on a C18 column and analysis in positive ion mode. The D2 and D3 forms are measured separately; results are reported as D2, D3, and the sum.

Vitamin E was measured quantitatively by LC-MS/MS. D6-alpha-tocopherol internal standard is added to serum, and proteins are precipitated with acetonitrile. The supernatant is subjected to online turbulent flow for sample cleanup, separated on a C18 column, and analyzed in positive ion mode.

Serum ferritin was measured quantitatively with a Beckman Coulter Access two-site immunoenzymatic (sandwich) assay. Serum ferritin binds mouse anti-ferritin that is immobilized on paramagnetic particles; ferritin is also bound by a goat anti-ferritin—alkaline phosphatase conjugate. After washing, alkaline phosphatase activity on a chemiluminescent substrate is measured and compared against a multi-point calibration curve of known ferritin concentrations.

MTHFR mutation analysis was performed for the A1298C and C677T variants using Hologic Invader assays. DNA was isolated from whole blood and amplified in the presence of probes for both wildtype and variant sequences. Hybridization of sequence-specific probes to genomic DNA leads to enzymatic cleavage of the probe, releasing an oligonucleotide that binds to a fluorescently labeled cassette. This second hybridization results in generation of a fluorescent signal that is specific to the wildtype or variant allele.

Sample preparation for measurement of plasma methylation and oxidative stress metabolites. For concentration determination of total thiols (homocysteine, cysteine, cyseinyl-glycine, glutamyl-cysteine, and glutathione), the disulfide bonds were reduced and protein-bond thiols were released by the addition of 50 μl freshly prepared 1.43 M sodium borohydride solution containing 1.5 μM EDTA, 66 mM NaOH and 10 n-amyl alcohol and added to 200 μl of plasma. After gentle mixing, the solution was incubated at +4° C. for 30 min with gentle shaking. To precipitate proteins, 250 μl ice cold 10% meta-phosphoric acid was added and the sample was incubated for 20 min on ice. After centrifugation at 18,000 g for 15 min at 4° C., the supernatant was filtered through a 0.2 μm nylon filter and a 20 μl aliquot was injected into the HPLC system.

For determination of free thiols and methylation metabolites, proteins were precipitated by the addition of 250 μl ice cold 10% meta-phosphoric acid and the sample was incubated for 10 min on ice. Following centrifugation at 18,000 g for 15 min at +4° C., the supernatant was filtered through a 0.2 μm nylon and a 20 μl aliquot was injected into the HPLC system.

HPLC with Coulometric Electrochemical Detection. The analyses were accomplished using HPLC with a Shimadzu solvent delivery system (ESA model 580) and a reverse phase C18 column (5 μm; 4.6×150 mm, MCM, Inc., Tokyo, Japan) obtained from ESA, Inc. (Chemsford, Mass.). A 20 aliquot of plasma extract was directly injected onto the column using Beckman autosampler (model 507E). All plasma metabolites were quantified using a model 5200A Coulochem II electrochemical detector (ESA, Inc., Chelmsford, Mass.) equipped with a dual analytical cell (model 5010) and a guard cell (model 5020). The concentrations of plasma metabolites were calculated from peak areas and standard calibration curves using HPLC software.

Metabolon Inc. Metabolon Inc. conducted measurements of metabolites in whole blood samples in a manner similar to a previous study. Briefly, individual samples were subjected to methanol extraction then split into aliquots for analysis by ultrahigh performance liquid chromatography/mass spectrometry (UHPLC/MS). The global biochemical profiling analysis comprised of four unique arms consisting of reverse phase chromatography positive ionization methods optimized for hydrophilic compounds (LC/MS Pos Polar) and hydrophobic compounds (LC/MS Pos Lipid), reverse phase chromatography with negative ionization conditions (LC/MS Neg), as well as a HILIC chromatography method coupled to negative (LC/MS Polar). All of the methods alternated between full scan MS and data dependent MSn scans. The scan range varied slightly between methods but generally covered 70-1000 m/z.

Metabolites were identified by automated comparison of the ion features in the experimental samples to a reference library of chemical standard entries that included retention time, molecular weight (m/z), preferred adducts, and in-source fragments as well as associated MS spectra and curated by visual inspection for quality control using software developed at Metabolon. Identification of known chemical entities was based on comparison to metabolomic library entries of purified standards.

### Statistical Analysis.

Univariate Analysis. To conduct a univariate analysis, a test was performed for whether the population means or medians between two populations are equal against the alternative hypothesis that they are not. To determine which testing method to use, the Anderson-Darling test was applied to each sample. If the recorded samples of a particular metabolite or ratio were drawn from two normal distributions an F-test was subsequently performed to determine whether the population variances of both distributions were identical. If at least one of the two samples of a particular metabolite or ratio was not drawn from a normal distribution, the two-sample Kolmogorov-Smirnov test was applied to examine whether the two samples were drawn from unknown distributions that had the same shape. This pre-analysis yielded four distinct scenarios for a particular metabolite or ratio: (i) both samples were drawn from normal distributions that had identical population variances, (ii) both samples were drawn from a normal distribution with unequal population variances, (iii) both samples were drawn from two unknown distributions that had the same shape and (iv) both samples were drawn from distinctively different distributions. For scenarios (i), (ii), (iii) and (iv) the standard Student t-test, the Welch test, the Mann-Whitney U test, and the Welch t-test were applied, respectively. A significance, α, of 0.05 was used. If a p-value was above α, the measurement is considered to have a significant difference between the population means or medians of the two groups. If a p-value was below α, the measurement is not considered significant. For scenario (iv), the result of the hypothesis test was declared as undetermined if the p-value was close to the significance α, e.g. if p=0.17, the hypothesis test is declared as undetermined.

In order to determine the robustness of the hypothesis tests, the false discovery rates (FDR) for each metabolite were also calculated. This was done by calculating the p-values for various combinations of mothers and calculating the fraction of p-values that were considered significant (≤0.05) over the total number of p-values. These combinations included every combination leaving one mother out each time, every combination leaving two mothers out at each time, and every combination leaving three mothers out at each time. This led to 1,770 p-values calculated for each metabolite from which the FDR was computed.

The area under the curve (AUC) of the receiver operating characteristic (ROC) curve was also calculated for each metabolite. The ROC curve is a plot of false positive rate (FPR) vs. the true positive rate (TPR). The higher the area under the curve is, the better the measurements are at classifying between the two groups of mothers.

A test was considered significant if the p-value was less than or equal to 0.05 and the FDR value was less than or equal to 0.1.

Multivariate Analysis. While the univariate analyses focused on testing for equal population means or medians of individual metabolites/ratios, this does not answer the question of how important the differences in mean or median are to separate the two groups of mothers. In order to examine the extent of the differences within the recorded observations of two samples, Fisher Discriminant Analysis (FDA) was applied. This technique defines a projection direction in the data space such that the squared difference between the centers of the projected observations of both samples over the variances of the projected observations is a maximum. Statistically, this objective function, J, is as follows:

$$J = \frac{{( {\overline{t}}_{1} - {\overline{t}}_{2} )}^{2}}{{s}_{1}^{2} + {s}_{2}^{2}} \qquad ( Eq . 1 )$$

Here,

$${\overline{t}}_{1} = \frac{1}{{n}_{1}} {\sum }_{i = 1}^{{n}_{1}} {t}_{1 , i} and {\overline{t}}_{2} = \frac{1}{{n}_{2}} {\sum }_{i = 1}^{{n}_{2}} {t}_{2 , i}$$

are the orthogonally projected means of both samples onto the direction vector and the sample variances of the projected data points are

$${s}_{1}^{2} = \frac{1}{{n}_{1} - 1} {\sum }_{i = 1}^{{n}_{1}} {( {t}_{1 , i} - {\overline{t}}_{1} )}^{2} and$$

$${s}_{2}^{2} = \frac{1}{{n}_{2 - 1}} {\sum }_{i = 1}^{{n}_{2}} {( {t}_{2 , i} - {\overline{t}}_{2} )}^{2} .$$

The orthogonal projection of i-th observation from the second sample, x_{2,i}, is t_{2,i}=x_{2,i}^{T}p, where p is the unit-length direction vector. Note that the projection coordinate, t_{2,i}, is often referred to as a score. Essentially, FDA is designed to best separate two groups of data while minimizing the spread of the data within each group. FDA is used to develop a multivariate model that can be used to classify between the two groups of data.

FDA works well with data consisting of real numbers. However, some of the data was discrete in nature such as the information about MTHFR gene mutation. For classification tasks including continuous and discrete data, logistic regression was used. Logistic regression is similar to linear regression, but the output is a binomial variable, or multinomial in the case of multiple classes. The output is the probability that a sample belongs to one class or another. The class that produces the highest probability is considered the class that the model classified the sample as belonging to.

The multivariate analysis made use of both FDA and logistic regression. The data was split into multiple subsets for analysis. These subsets include: (i) the 20 measurements from the FOCM/TS pathways, (ii) the metabolites from the FOCM/TS pathways plus additional nutritional information, (iii) the FOCM/TS metabolites with the additional nutritional information and the MTHFR gene information, and (iv) the FOCM/TS metabolites with additional nutritional information and the MTHFR gene information and a select number of significant metabolites from the broad metabolomics analysis. The metabolites selected from the Metabolon dataset to be included in analysis were the 50 metabolites with the highest AUC for the ROC in order to reduce the number of metabolites used for analysis from 621 to 76. All combinations of two through ten variables were analyzed in each subset. FDA was used for subsets (i) and (ii) and logistic regression was used for subsets (iii) and (iv) because subsets (iii) and (iv) contained the MTHFR gene information which was a binary variable. The analysis included these different subtasks, instead of just investigating the full set of measurements, to be able to see if differences in the FOCM/TS pathways previously found in pregnant women who have had a child with ASD would apply to women who are not pregnant and to determine what other information may be important to classify between the two groups of mothers.

In order to determine the most significantly contributing metabolites out of the set of metabolites being analyzed, all combinations involving two through ten metabolites were studied. This evaluation entailed the use of a leave-one-out cross-validatory procedure. Leave-one-out cross-validation removes the first observation, determining a model using (Eq. 1) and the n−1 variables, and then applying this model to the first observation. This application is designed to determine whether this observation is correctly/incorrectly classified as belonging to group 1 or 2. Then, the second observation is left out, whilst the first observation is included for determining a second model using (Eq. 1). The second model is then also used to decide whether the second observation is correctly classified or misclassified. Repeating this procedure until each of the observations is left out once allows the calculation of the overall rate of correctly classified and misclassified observations. For determining whether an observation is correctly or incorrectly classified, the samples describing the ASD group were defined as positives and the corresponding samples of the Typically Developing (TD) cohort as negatives. Hypothesis testing was performed to test if an observation belongs to a cohort and the one-sided acceptance regions for a significance of α=0.05 was determined on the basis of a kernel density estimation of the scores for the observations of the cohort. This allowed the calculation of the number of true and false positives as well as the number of true and false negatives for the observations left out, i.e. independently, and with it the accuracy, specificity and sensitivity metrics and the confusion matrix. The optimum combination of metabolites was determined to be the one producing the lowest errors.

### Results.

This section provides information about the study participants, the results of the univariate analysis of the metabolites, the multi-variate analyses for the 4 subsets of metabolites discussed in the methodology section, and lastly a correlation analysis to investigate the grouping of metabolites into five primary groups.

### Participants.

Thirty mothers who have a child with ASD (ASD-M) and 29 mothers who have a typically-developing children (TD-M) were recruited for this study. The average age of mothers in the ASD-M group was 35.4 years as compared to 34.9 years for the TD-M group. Similarly, the average ages of their children were 4.71 and 3.87 years for the ASD-M and TD-groups, respectively. A more detailed breakdown of the characteristics of the participants is shown in Table 19.

TABLE 19
Characteristics of the study participants. The p-value was calculated
for each characteristic to test for significant differences between
the two groups. The p-value was calculated using the t-test for
the numerical variables and Chi-Squared for the categorical variables.
If the Chi-Squared test was used C was added to the result and
if a t-test was used, T was added to the result. If the p-value
was greater than 0.05, the p-value was marked as n.s. for not
significant. The FDR was calculated for the variables with significant
differences in the two groups.

| | | | p-value of | |
|---|---|---|---|---|
| | | | T-test (T) or | |
| | ASD-M | TD-M | Chi-Squared | |
| | (n = 30) | (n = 29) | (C) | FDR |
| Maternal age | 35.4 | 34.9 | n.s.T | |
| Child gender | 22 m, 8 f (73% | 14 m, 14 f | 0.05C | 0.6268 |
| | male) | (50% male) | | |
| Child age | 4.71 (1.0) | 3.87 (1.3) | 0.0091T | 0.00 |
| Pregnancy | 43% | 39% | n.s.C | |
| complica- | (18% mild, 18% | (25% mild, | | |
| tions | moderate, 7% | 14% | | |
| | severe | moderate, 0% | | |
| | | severe) | | |
| Birth | 50% | 32% | n.s.C | |
| complica- | (36% mild, 11% | (21% mild, 7% | | |
| tions | moderate, 4% | moderate, 4% | | |
| | severe) | severe) | | |
| C-section | 43% | 29% | n.s.C | |

Most of the characteristics of the study participants were well-matched between the two groups of mothers. The only characteristic listed here with a significant difference between the two groups is the age of their children.

### Univariate Analysis.

FOCM/TS Metabolites. (folate-dependent one carbon metabolism (FOCM) and transsulfuration (TS)) The univariate results for the FOCM/TS metabolites are shown in Table 9. Levels of vitamin B12 and the SAM/SAH ratio are significantly lower in the ASD-M group compared to the TD-M group, (p≤0.05, FDR≤0.1). Also, levels of Glu-Cys, fCysteine, and fCystine are significantly higher in the ASD-M group compared to the TD-M group (p≤0.05, FDR≤0.1).

TABLE 9
Univariate results for FOCM/TS metabolites and vitamin E, folate, ferritin,
B12, MMA, and MTHFR status. The measurements are ordered by decreasing AUC.
Statistically significant metabolites with p-value ≤ 0.05 and FDR ≤
0.1 are shown in gray and * indicates measurements that were left out of the
classification procedure as the measurements were not collected from all mothers.
Specifically, these were Vitamin D with 28 mothers in ASD-M and 28 TD-M mothers
and Isoprostane with 28 participants that were ASD-M and 25 mothers in TD-M.

| | | ASD-M | TD-M | | | | Ratio |
|---|---|---|---|---|---|---|---|
| | | (mean ± std) | (mean ± std) | p- | | | (ASD-M/ |
| Metabolite | Test | N = 30 | N = 29 | Values | FDR | AUC | TD-M) |
| B12 | MW | 355 ± 196 | 473 ± 173 | 2.40E−03 | 0.00 | 0.73 | 0.75 |
| fCysteine | t= | 23.8 ± 1.88 | 22.6 ± 1.94 | 0.01 | 0.00 | 0.70 | 1.06 |
| Glu-Cys | t= | 1.89 ± 0.22 | 1.72 ± 0.24 | 0.01 | 0.00 | 0.69 | 1.10 |
| SAM/SAH | t= | 1.94 ± 0.25 | 2.09 ± 0.20 | 0.01 | 0.00 | 0.67 | 0.93 |
| fCystine | t= | 24.1 ± 2.78 | 22.4 ± 2.43 | 0.02 | 0.01 | 0.67 | 1.07 |
| tCysteine | t= | 248 ± 23.1 | 234 ± 28.3 | 0.04 | 0.40 | 0.64 | 1.06 |
| tGSH | t= | 6.23 ± 0.98 | 5.85 ± 1.07 | 0.15 | 1.00 | 0.63 | 1.07 |
| SAM | t= | 47.2 ± 5.37 | 49.3 ± 5.76 | 0.15 | 1.00 | 0.62 | 0.96 |
| Methionine | t= | 19.9 ± 2.56 | 20.8 ± 2.98 | 0.19 | 1.00 | 0.61 | 0.95 |
| MTHFR mut. | χ² | | | 0.15 | 1.00 | 0.60 | |
| (A1298C) | | | | | | | |
| tGSH/GSSG | t= | 29.5 ± 6.21 | 27.3 ± 6.06 | 0.18 | 1.00 | 0.60 | 1.08 |
| Folate | MW | 17.6 ± 6.19 | 21.1 ± 9.17 | 0.20 | 1.00 | 0.60 | 0.83 |
| Homocysteine | MW | 8.63 ± 0.98 | 8.27 ± 1.18 | 0.21 | 1.00 | 0.60 | 1.04 |
| tGSH/GSSG | t= | 29.5 ± 6.21 | 27.3 ± 6.06 | 0.18 | 1.00 | 0.60 | 1.08 |
| SAH | t= | 24.5 ± 2.66 | 23.6 ± 2.04 | 0.15 | 1.00 | 0.59 | 1.04 |
| Vitamin D3* | t= | 27.1 ± 9.10 | 24.9 ± 6.08 | 0.27 | 1.00 | 0.57 | 1.09 |
| Ferritin | MW | 35.1 ± 31.0 | 29.5 ± 26.1 | 0.36 | 1.00 | 0.57 | 1.19 |
| Cys-Gly | t= | 38.7 ± 5.06 | 37.6 ± 6.38 | 0.46 | 1.00 | 0.57 | 1.03 |
| Adenosine | t= | 0.22 ± 0.03 | 0.21 ± 0.03 | 0.28 | 1.00 | 0.55 | 1.04 |
| fGSH/GSSG | MW | 8.73 ± 2.08 | 8.81 ± 1.84 | 0.49 | 1.00 | 0.55 | 0.99 |
| % Oxidized | MW | 0.19 ± 0.03 | 0.19 ± 0.04 | 0.49 | 1.00 | 0.55 | 1.01 |
| Glutathione | | | | | | | |
| Chlorotyrosine | t= | 26.8 ± 4.27 | 27.6 ± 4.23 | 0.51 | 1.00 | 0.55 | 0.97 |
| Nitrotyrosine | t≠ | 32.9 ± 6.28 | 33.7 ± 4.67 | 0.59 | 1.00 | 0.55 | 0.98 |
| fGSH | t= | 1.85 ± 0.32 | 1.89 ± 0.35 | 0.62 | 1.00 | 0.55 | 0.98 |
| fCystine/fCysteine | t= | 1.01 ± 0.11 | 1.00 ± 0.10 | 0.59 | 1.00 | 0.54 | 1.02 |
| Vitamin E | t= | 9.23 ± 2.53 | 9.82 ± 3.22 | 0.75 | 1.00 | 0.54 | 0.94 |
| Isoprostane (U)* | MW | 0.15 ± 0.10 | 0.18 ± 0.14 | 0.70 | 1.00 | 0.53 | 0.83 |
| Isoprostane (U)* | MW | 0.15 ± 0.10 | 0.18 ± 0.14 | 0.70 | 1.00 | 0.53 | 0.83 |
| MTHFR mut. | χ² | | | 0.90 | 1.00 | 0.53 | |
| (C677T) | | | | | | | |
| GSSG | t= | 0.22 ± 0.04 | 0.22 ± 0.03 | 0.93 | 1.00 | 0.52 | 1.00 |
| MMA | MW | 0.15 ± 0.06 | 0.15 ± 0.08 | 0.64 | 1.00 | 0.51 | 0.96 |

Global metabolic profile-Metabolon. 622 metabolites were measured in whole blood. The univariate analysis for the 50 metabolites from broad metabolomics with the highest AUC values are shown in the Table 10. They are ordered starting with those with the highest AUC. Note that these are semi-quantitative measurements (no absolute values), so only the ratio of ASD-M/TD-M is shown. In almost every case the ASD-M group had lower levels of metabolites than the TD-M group, with the levels of 4-vinylphenol sulfate, NAD+, and three glycine-containing metabolites (gamma-glutamylglycine, cinnamoylglycine, propionylglycine) being especially low (ASD-M/TD-M ratio <0.50). Four metabolites were higher in the ASD-M group (histidylglutamate, asparaginylalanine, dimethyl sulfone, and mannose). Note that dimethyl sulfone was unusually high in the ASD-M group (ASD-M/TD-M ratio=18.7, p=0.01, but the FDR was not significant). 80% of the TD-M measurements of dimethyl sulfone and 47% of the ASD-M measurements of dimethyl sulfone were below the detection limit, and the distribution of the data for is skewed.

TABLE 10
Univariate results of the 50 metabolites (measured by Metabolon)
from broad metabolomics with the highest area under the receiver
operating characteristic (ROC) curve (AUC). Metabolites with
p-value ≤ 0.05 and FDR ≤ 0.1 are shown in gray.

| | | | | | Ratio |
|---|---|---|---|---|---|
| | | | | | (ASD-M/ |
| Metabolite | Test | p-Value | FDR | AUC | TD-M) |
| Fructose | t≠* | 6.88E−04 | 0.00 | 0.81 | 0.60 |
| Histidylglutamate | t≠ | 2.67E−05 | 0.00 | 0.80 | 1.50 |
| Decanoylcarnitine (C10) | t≠ | 1.55E−04 | 0.00 | 0.78 | 0.66 |
| S-1-pyrroline-5-carboxylate | MW | 3.09E−04 | 0.00 | 0.77 | 0.63 |
| Octanoylcarnitine (C8) | MW | 4.00E−04 | 0.00 | 0.77 | 0.67 |
| 4-vinylphenol sulfate | t≠* | 1.30E−03 | 0.00 | 0.77 | 0.31 |
| Cis-4-decenoylcarnitine (C10:1) | t= | 5.82E−04 | 0.00 | 0.74 | 0.75 |
| N-formylanthranilic acid | t= | 1.20E−03 | 0.00 | 0.74 | 0.69 |
| N-acetylasparagine | t≠* | 1.90E−03 | 0.00 | 0.73 | 0.78 |
| Arachidoylcarnitine (C20)* | MW | 3.00E−03 | 0.00 | 0.73 | 0.85 |
| N-palmitoylglycine | t= | 2.20E−03 | 0.00 | 0.72 | 0.81 |
| Citrulline | t= | 2.60E−03 | 0.00 | 0.72 | 0.91 |
| 6-hydroxyindole sulfate | t≠ | 1.20E−03 | 0.00 | 0.72 | 0.59 |
| N-palmitoylserine | MW | 4.00E−03 | 0.00 | 0.72 | 0.77 |
| Myristoylcarnitine (C14) | MW | 4.10E−03 | 0.00 | 0.72 | 0.82 |
| Laurylcarnitine (C12) | MW | 4.30E−03 | 0.00 | 0.72 | 0.74 |
| Stearoylcarnitine (C18) | MW | 0.01 | 0.00 | 0.71 | 0.85 |
| Gamma-glutamylglycine | t≠* | 2.20E−03 | 0.00 | 0.71 | 0.27 |
| 5-oxoproline | t= | 0.01 | 0.00 | 0.70 | 0.94 |
| Asparaginylalanine | t= | 3.90E−03 | 0.00 | 0.70 | 1.32 |
| Glutamine | t= | 0.02 | 0.00 | 0.70 | 0.95 |
| Catechol sulfate | MW | 0.01 | 0.00 | 0.70 | 0.67 |
| 3-indoxyl sulfate | t≠ | 2.70E−03 | 0.00 | 0.70 | 0.67 |
| 7-methylxanthine | MW | 0.01 | 0.00 | 0.70 | 0.58 |
| Phenol sulfate | MW | 0.01 | 0.00 | 0.70 | 0.67 |
| Cinnamoylglycine | t≠* | 0.01 | 0.00 | 0.70 | 0.46 |
| Alpha-ketoglutaramate* | t= | 0.02 | 0.00 | 0.70 | 0.73 |
| Isovalerylglycine | MW | 0.01 | 0.00 | 0.69 | 0.78 |
| Propionylglycine | MW | 0.01 | 0.00 | 0.69 | 0.48 |
| Docosapentaenoylcarnitine | MW | 0.01 | 0.00 | 0.69 | 0.72 |
| (C22:5n3)* | | | | | |
| N-acetyl-2-aminooctanoate* | t≠ | 3.20E−03 | 0.00 | 0.69 | 0.54 |
| S-methylglutathione | t= | 0.02 | 0.01 | 0.69 | 0.86 |
| Gamma-glutamyltyrosine | MW | 0.02 | 0.00 | 0.68 | 0.64 |
| Succinylcarnitine (C4-DC) | t= | 0.03 | 0.07 | 0.68 | 0.87 |
| Arachidonoylcarnitine (C20:4) | MW | 0.02 | 0.00 | 0.68 | 0.77 |
| Glycine | t= | 0.01 | 0.00 | 0.68 | 0.87 |
| N-acetylvaline | t= | 0.04 | 0.28 | 0.68 | 0.80 |
| Lignoceroylcarnitine (C24)* | t= | 0.02 | 0.01 | 0.68 | 0.84 |
| Guaiacol sulfate | MW | 0.02 | 0.01 | 0.68 | 0.81 |
| 5-methylthioadenosine (MTA) | MW | 0.02 | 0.00 | 0.68 | 0.86 |
| Proline | MW | 0.02 | 0.00 | 0.68 | 0.90 |
| Pyridoxate | MW | 0.02 | 0.00 | 0.68 | 0.75 |
| Palmitoylcarnitine (C16) | MW | 0.02 | 0.03 | 0.67 | 0.83 |
| Eicosenoylcarnitine (C20:1)* | MW | 0.02 | 0.05 | 0.67 | 0.83 |
| Nicotinamide adenine dinucleotide | t≠* | 0.03 | 0.05 | 0.67 | 0.41 |
| (NAD+) | | | | | |
| Dimethyl sulfone | MW | 0.01 | 0.23 | 0.67 | 18.7 |
| Tiglylcarnitine (C5:1-DC) | MW | 0.02 | 0.02 | 0.67 | 0.63 |
| Adrenoylcarnitine (C22:4)* | MW | 0.03 | 0.11 | 0.67 | 0.74 |
| 3-methylxanthine | MW | 0.03 | 0.13 | 0.67 | 0.74 |
| Mannose | MW | 0.03 | 0.19 | 0.67 | 1.21 |

Hypothesis testing was also done on the entire Metabolon dataset and revealed that 48 of these metabolites had significant differences between the two groups of mothers. There were 3 metabolites not included in the top 50 from this set that showed significant differences between mean/median between the two groups, but these were not included in the multivariate analysis because they had lower AUC values than the metabolites included (see Table 20).

TABLE 20
Metabolites from the Metabolon dataset not included in the top
50 for analysis with significant p-values and FDR values. The
p-Values, FDR values, and AUC values are listed in the table
and sorted by AUC value (largest to smallest), p-value (smallest
to largest), and then FDR value (smallest to largest).

| Metabolite | Test | p-Value | FDR | AUC |
|---|---|---|---|---|
| 2-hydroxyphenylacetate | t= | 0.02 | 0.02 | 0.66 |
| N-acetylleucine | MW | 0.02 | 0.02 | 0.66 |
| Margaroylcarnitine (C17)* | t≠ | 0.02 | 0.04 | 0.65 |

Table 11 contains more information about the metabolites in Table 10. Table 11 lists the many metabolic pathways which had significant differences between the ASD-M and TD-M groups, including amino acids (15 metabolites), carbohydrates (1), vitamins (2), energy (1), lipids (16), peptides (4), and xenobiotics (7). When considering sub-pathways, there were differences in alanine/aspartate metabolism (1 metabolite), glutamate metabolism (3), glutathione metabolism (2), glycine (1), leucine/isoleucine/valine (3), polyamine (1), tryptophan (2), tyrosine (1), urea cycle (2), fructose/mannose (2), nicotinamide (1), vitamin B6 (1), vitamin B12 (1), TCA cycle (1), endocannabinoid (1), carnitine/fatty acid metabolism (12), other fatty acid metabolism (3), dipeptides (2), gamma-glutamyl (2), benzoate (3), chemical/xenobiotics (2), cinnamoylglycine (1), and xanthine metabolism (2).

TABLE 11
Pathways and subpathways of the 50 metabolites from the broad
metabolomics data with the highest area under the ROC curve (AUC)
sorted by pathway and subpathway. A fourth column lists whether the
metabolites were higher or lower in the ASD-M group. Metabolites
that had a p-value ≤ 0.05 and FDR ≤ 0.1 (FDR-values
listed in Table 10) are shown in gray.

| | | | Higher/ |
|---|---|---|---|
| | | | lower in |
| | | | ASD-M |
| Metabolite | Pathway | Sub-Pathway | group |
| N-acetylasparagine | Amino Acid | Alanine and | ↓ |
| | | Aspartate | |
| | | Metabolism | |
| S-1-pyrroline-5- | Amino Acid | Glutamate | ↓ |
| carboxylate | | Metabolism | |
| Glutamine | Amino Acid | Glutamate | ↓ |
| | | Metabolism | |
| Alpha-ketoglutaramate* | Amino Acid | Glutamate | ↓ |
| | | Metabolism | |
| 5-oxoproline | Amino Acid | Glutathione | ↓ |
| | | Metabolism | |
| S-methylglutathione | Amino Acid | Glutathione | ↓ |
| | | Metabolism | |
| Glycine | Amino Acid | Glycine, Serine and | ↓ |
| | | Threonine | |
| | | Metabolism | |
| Isovalerylglycine | Amino Acid | Leucine, Isoleucine | ↓ |
| | | and Valine | |
| | | Metabolism | |
| N-acetylvaline | Amino Acid | Leucine, Isoleucine | ↓ |
| | | and Valine | |
| | | Metabolism | |
| Tiglylcarnitine | Amino Acid | Leucine, Isoleucine | ↓ |
| (C5:1-DC) | | and Valine | |
| | | Metabolism | |
| 5-methylthioadenosine | Amino Acid | Polyamine | ↓ |
| (MTA) | | Metabolism | |
| N-formylanthranilic | Amino Acid | Tryptophan | ↓ |
| acid | | Metabolism | |
| 3-indoxyl sulfate | Amino Acid | Tryptophan | ↓ |
| | | Metabolism | |
| Phenol sulfate | Amino Acid | Tyrosine | ↓ |
| | | Metabolism | |
| Citrulline | Amino Acid | Urea cycle; Arginine | ↓ |
| | | and Proline | |
| | | Metabolism | |
| Proline | Amino Acid | Urea cycle; Arginine | ↓ |
| | | Proline Metabolism | |
| Mannose | Carbohy- | Fructose, Mannose | ↑ |
| | drate | and Galactose | |
| | | Metabolism | |
| Fructose | Carbohy- | Fructose, Mannose, | ↓ |
| | drate | and Galactose | |
| | | Metabolism | |
| Nicotinamide adenine | Cofactors | Nicotinate and | ↓ |
| dinucleotide (NAD+) | and Vitamins | Nicotinamide | |
| | | Metabolism | |
| Pyridoxate | Cofactors | Vitamin B6 | ↓ |
| | and Vitamins | Metabolism | |
| Succinylcarnitine | Energy | TCA Cycle | ↓ |
| (C4-DC) | | | |
| N-palmitoylserine | Lipid | Endocannabinoid | ↓ |
| Decanoylcarnitine (C10) | Lipid | Fatty Acid | ↓ |
| | | Metabolism (Acyl | |
| | | Carnitine) | |
| Octanoylcarnitine (C8) | Lipid | Fatty Acid | ↓ |
| | | Metabolism (Acyl | |
| | | Carnitine) | |
| Cis-4-decenoylcarnitine | Lipid | Fatty Acid | ↓ |
| (C10:1) | | Metabolism (Acyl | |
| | | Carnitine) | |
| Arachidoylcarnitine | Lipid | Fatty Acid | ↓ |
| (C20)* | | Metabolism (Acyl | |
| | | Carnitine) | |
| Myristoylcarnitine (C14) | Lipid | Fatty Acid | ↓ |
| | | Metabolism (Acyl | |
| | | Carnitine) | |
| Laurylcarnitine (C12) | Lipid | Fatty Acid | ↓ |
| | | Metabolism (Acyl | |
| | | Carnitine) | |
| Stearoylcarnitine (C18) | Lipid | Fatty Acid | ↓ |
| | | Metabolism (Acyl | |
| | | Carnitine) | |
| Docosapentaenoylcarni- | Lipid | Fatty Acid | ↓ |
| tine (C22:5n3)* | | Metabolism (Acyl | |
| | | Carnitine) | |
| Arachidonoylcarnitine | Lipid | Fatty Acid | ↓ |
| (C20:4) | | Metabolism (Acyl | |
| | | Carnitine) | |
| Lignoceroylcarnitine | Lipid | Fatty Acid | ↓ |
| (C24)* | | Metabolism (Acyl | |
| | | Carnitine) | |
| Palmitoylcarnitine | Lipid | Fatty Acid | ↓ |
| (C16) | | Metabolism (Acyl | |
| | | Carnitine) | |
| Eicosenoylcarnitine | Lipid | Fatty Acid | ↓ |
| (C20:1)* | | Metabolism (Acyl | |
| | | Carnitine) | |
| Adrenoylcarnitine | Lipid | Fatty Acid | ↓ |
| (C22:4)* | | Metabolism (Acyl | |
| | | Carnitine) | |
| N-palmitoylglycine | Lipid | Fatty Acid | ↓ |
| | | Metabolism (Acyl | |
| | | Glycine) | |
| Propionylglycine | Lipid | Fatty Acid | ↓ |
| | | Metabolism (also | |
| | | BCAA Metabolism) | |
| N-acetyl-2- | Lipid | Fatty Acid, Amino | ↓ |
| aminooctanoate* | | | |
| Histidylglutamate | Peptide | Dipeptide | ↑ |
| Asparaginylalanine | Peptide | Dipeptide | ↑ |
| Gamma-glutamylglycine | Peptide | Gamma-glutamyl | ↓ |
| | | Amino Acid | |
| Gamma-glutamyltyrosine | Peptide | Gamma-glutamyl | ↓ |
| | | Amino Acid | |
| 4-vinylphenol sulfate | Xenobiotics | Benzoate Metabolism | ↓ |
| Catechol sulfate | Xenobiotics | Benzoate Metabolism | ↓ |
| Guaiacol sulfate | Xenobiotics | Benzoate Metabolism | ↓ |
| 6-hydroxyindole sulfate | Xenobiotics | Chemical | ↓ |
| Dimethyl sulfone | Xenobiotics | Chemical | ↑ |
| Cinnamoylglycine | Xenobiotics | Food | ↓ |
| | | Component/Plant | |
| 7-methylxanthine | Xenobiotics | Xanthine Metabolism | ↓ |
| 3-methylxanthine | Xenobiotics | Xanthine Metabolism | ↓ |

Carnitine. As shown in Table 12, several carnitine-conjugated metabolites are significantly different in the two groups of mothers. Table 12 below highlights the univariate hypothesis testing results for the carnitine-conjugated metabolites specifically in order of increasing size, from 4-carbon to 24 carbon chains. The ratio of ASD/TD for carnitine-conjugated metabolites was consistently low, ranging from 0.63 to 0.87, with an average of 0.77. There were 33 additional carnitine metabolites in the 600 metabolites measured by untargeted metabolomics. Of these 33, only three had ratios indicating levels of the carnitine were higher in the ASD-M group than in the TD-M group. Also, eight of these metabolites showed significant difference in mean/median between the two groups using hypothesis testing. All of the eight carnitine metabolites had ratios indicating that the levels of carnitine-conjugated molecules in the ASD-M group were less than in the TD-M group.

TABLE 12
Univariate hypothesis testing results for the carnitine-conjugated
metabolites. Statistically significant metabolites with
a p-value ≤ 0.05 and FDR ≤ 0.1 are shown in gray.

| | | | | | Ratio |
|---|---|---|---|---|---|
| | | | | | (ASD-M/ |
| Carnitine | Test | p-Value | FDR | AUC | TD-M |
| Succinylcarnitine (C4-DC) | t= | 0.03 | 0.07 | 0.68 | 0.87 |
| Tiglylcarnitine (C5:1-DC) | MW | 0.02 | 0.02 | 0.67 | 0.63 |
| Octanoylcarnitine (C8) | MW | 4.00E−04 | 0.00 | 0.77 | 0.67 |
| Decanoylcarnitine (C10) | t≠ | 1.55E−04 | 0.00 | 0.78 | 0.66 |
| Cis-4-decanoylcarnitine | t= | 5.82E−04 | 0.00 | 0.74 | 0.75 |
| (C10:1) | | | | | |
| Laurylcarnitine (C12) | MW | 4.30E−03 | 0.00 | 0.72 | 0.74 |
| Myristoylcarnitine (C14) | MW | 4.10E−03 | 0.00 | 0.72 | 0.82 |
| Palmitoylcarnitine (C16) | MW | 0.02 | 0.03 | 0.67 | 0.83 |
| Arachidoylcarnitine (C20)* | MW | 3.00E−03 | 0.00 | 0.73 | 0.85 |
| Eicosenoylcarnitine | MW | 0.02 | 0.05 | 0.67 | 0.83 |
| (C20:1)* | | | | | |
| Arachidonoylcarnitine | MW | 0.02 | 0.00 | 0.68 | 0.77 |
| (C20:4) | | | | | |
| Adrenoylcarnitine (C22:4)* | MW | 0.03 | 0.11 | 0.67 | 0.74 |
| Docosapentaenoylcarnitine | MW | 0.01 | 0.00 | 0.69 | 0.72 |
| (C22:5n3)* | | | | | |
| Lignoceroylcarnitine | t= | 0.02 | 0.01 | 0.68 | 0.84 |
| (C24)* | | | | | |

### Multivariate Analysis.

The multivariate analysis was performed using multiple subsets of data. The subsets included the twenty metabolites from the FOCM/TS pathways (i), the FOCM/TS metabolites plus some additional nutritional information (ii), the FOCM/TS metabolites plus the additional nutritional information and the MTHFR gene information (iii), and subset (iii) plus fifty metabolites from the broad metabolomics analysis (iv). The first two subsets were analyzed using FDA because all of the variables were continuous and the last two subsets were analyzed using logistic regression because the variables included both continuous and binary data. Each multivariate analysis was combined with leave-one-out cross-validation in order to analyze the success of the model on classification. The best combinations of metabolites from each of the first three subsets had errors ranging from 20-27%. Table 13 below details the type I/type II errors using these metabolites.

TABLE 13
Multivariate results for combination the best combination
of metabolites from the first three subsets (i-
iii) with lowest type I/type II errors.

| | | Type I | Type II |
|---|---|---|---|
| Subset | Combination | Error | Error |
| (i): FOCM/TS | tCysteine, Glu-Cys, | 24% | 27% |
| Metabolites | fCysteine, | | |
| | fCystine/fCystiene, | | |
| | Nitrotyrosine | | |
| (ii): FOCM/TS | SAM/SAH, Glu-Cys, GSSG, | 24% | 27% |
| metabolites plus | fCysteine, B12 | | |
| nutritional | | | |
| information | | | |
| (iii): FOCM/TS | SAM/SAH, tCysteine, | 24% | 20% |
| metabolites, | Glu-Cys, B12, MTHFR mut. | | |
| nutritional | (A1298C) | | |
| information, and | | | |
| MTHFR gene | | | |
| information | | | |

The highest accuracies were found when analyzing the fourth, and largest, subset of metabolites. The best combinations for 2, 3, 4, and 5 metabolites for subset iv are shown in Table 14; combinations that contained more than 5 variables resulted in a decrease in accuracy due to overfitting of the classification model. It is important to note that many other combinations of metabolites yielded similar results and the top combinations of five metabolites are listed in Table 14. The results for using even only two metabolites resulted in lower Type 1 and Type 2 errors than the analysis using the other subsets described above (Table 13) and including more than two metabolites for classification further improved accuracy.

TABLE 14
Multivariate results using top combinations
of 2-5 variables from subset (iv).

| | Type I | Type II |
|---|---|---|
| Metabolites | Error (FPR) | Error (FNR) |
| 2 metabolites: | 17% | 13% |
| Histidylglutamate, 6- | | |
| hydroxyindoel sulfate | | |
| 3 metabolites: | 7% | 7% |
| Histidylglutamate, N- | | |
| formylanthranilic acid, | | |
| palmitoylcarnitine (C16) | | |
| 4 metabolites: | 3% | 7% |
| Histidylglutamate, S-1- | | |
| pyrroline-5-carboxylate, N- | | |
| acetyl-2-aminooctanoate*, | | |
| 5-methylthioadenosine | | |
| (MTA) | | |
| 5 Metabolites: | 3% | 3% |
| Glu-Cys, histidylglutamate, | | |
| cinnamoylglycine, proline, | | |
| adrenoylcarnitine (C22:4)* | | |

TABLE 15
Multivariate results using the top combinations
of 5 variables from subset (iv).

| | Type I | Type II |
|---|---|---|
| Metabolites | Error (FPR) | Error (FNR) |
| SAM/SAH, percent oxidized, | 3% | 7% |
| histidylglutamate, cis-4- | | |
| decenoylcarnitine (C10:1), 3- | | |
| indoxyl sulfate | | |
| fGSH/GSSG, histidylglutamate, | 3% | 7% |
| 4-vinylphenol sulfate, 3-indroxyl | | |
| sulfate, palmitoylcarnitine (C16) | | |
| Histidylglutamate, 4-vinylphenol | 3% | 7% |
| sulfate, cinnamoylglycine, N- | | |
| acetylvaline, palmitoylcarnitine | | |
| (C16) | | |
| Glu-Cys, histidylglutamate, | 3% | 7% |
| catechol sulfate, phenol sulfate, | | |
| N-acetyl-2-aminooctanoate* | | |
| tGSH, 4-vinylphenol sulfate, 5- | 7% | 3% |
| oxoproline, asparaginylalanine, | | |
| tiglylcarnitine (C5:1-DC) | | |

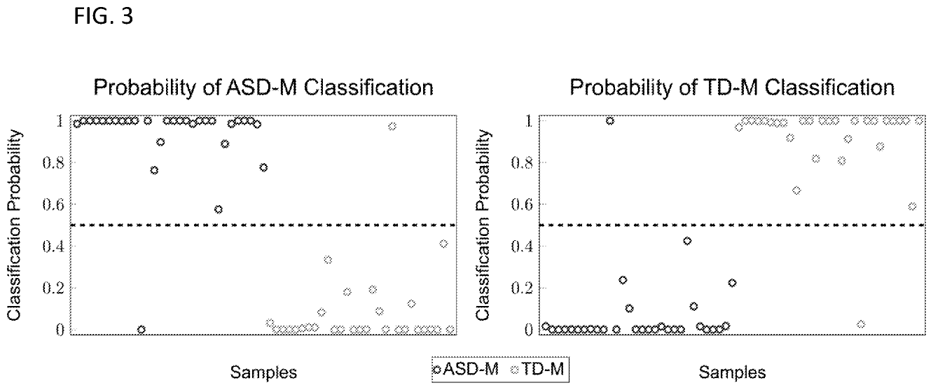

To further illustrate classification accuracy, the 5-metabolite model from Table 14 was used and the probabilities that the samples would be classified by the model in each of the two groups are shown in FIG. 3. The metabolites of this 5-metabolite model consisting of Glu-Cys, histidylglutamate, cinnamoylglycine, proline, adrenoylcarnitine (C22:4)* are hereafter referred to as the core metabolites.

The plots show that the ASD-M samples have a high probability of being classified as ASD-M and the TD-M samples have a high probability of being classified as TD-M. The results from this figure coupled with the low misclassification errors from Table 14 show that there are significant metabolic differences between the two groups of mothers and that these differences are sufficiently large to allow for accurate classification in the vast majority of cases.

In order to further investigate the differences between the two groups, we calculated the correlation coefficients between the 5 metabolites from the best classification model (Table 14) and the rest of the metabolites considered in the analysis for the combined set of ASD-M and TD-M samples. The metabolites that had the highest correlation coefficients with these metabolites are listed in Table 16. We also calculated the correlation of the top 5 metabolites with one another, and, as expected, found very little correlation among these five (see Table 17); this is not unexpected as the classification algorithms tries to identify metabolites that provide new information that can be used for classification as redundant information will not increase classification accuracy. This suggests that there are five general areas of metabolic differences in mothers of children with/without ASD involving 9 or more metabolites for each area.

TABLE 16
Correlation coefficients between the five metabolite model
from Table 13 that provides the highest classification
accuracy and the other 71 analyzed metabolites.

| | | |
|---|---|---|
| Metabolite | Correlation Coefficient | p-Value |

Glu-Cys

| | | |
|---|---|---|
| tGSH | 0.55 | 5.71E−06 |
| tGSH/GSSG | 0.35 | 0.01 |
| 6-hydroxyindole sulfate | −0.25 | 0.05 |
| SAM/SAH | −0.26 | 0.04 |
| N-formylanthranilic acid | −0.28 | 0.03 |
| 5-methylthioadenosine | −0.28 | 0.03 |
| (MTA) | | |
| Pyridoxate | −0.31 | 0.02 |
| Folate | −0.38 | 3.40E−03 |

Histidylglutamate

| | | |
|---|---|---|
| Asparaginylalanine | 0.55 | 6.74E−06 |
| Mannose | 0.40 | 1.70E−03 |
| fCystine | 0.30 | 0.02 |
| Succinylcarntine (C4-DC) | −0.27 | 0.04 |
| Citrulline | −0.27 | 0.04 |
| Fructose | −0.28 | 0.03 |
| Octanoylcarnitine (C8) | −0.29 | 0.02 |
| Gamma-glutamylglycine | −0.30 | 0.02 |
| Isovaleryglycine | −0.32 | 0.01 |
| Decanoylcarnitine (C10) | −0.33 | 0.01 |

Cinnamoylglycine

| | | |
|---|---|---|
| N-acetyl-2- | 0.45 | 4.13E−04 |
| aminooctanoate* | | |
| N-formylanthranilic acid | 0.44 | 4.30E−04 |
| 3-indoxyl sulfate | 0.35 | 0.01 |
| Citrulline | 0.33 | 0.01 |
| 6-hydroxyindole sulfate | 0.32 | 0.01 |
| Chlorotyrosine | 0.29 | 0.02 |
| Alpha-ketoglutaramate* | 0.28 | 0.03 |
| Nicotinamide adenine | 0.28 | 0.03 |
| dinucleotide (NAD+) | | |
| Pyridoxate | 0.27 | 0.04 |
| Guaiacol sulfate | 0.26 | 0.04 |
| S-methylglutathione | 0.26 | 0.04 |
| Methionine | −0.29 | 0.02 |
| fCysteine | −0.33 | 0.01 |

Proline

| | | |
|---|---|---|
| S-1-pyrroline-5-carboxylate | 0.59 | 1.22E−06 |
| Gamma-glutamyltyrosine | 0.45 | 3.73E−04 |
| 3-indoxyl sulfate | 0.44 | 5.33E−04 |
| 6-hydroxyindole sulfate | 0.43 | 6.01E−04 |
| Phenol sulfate | 0.41 | 1.20E−03 |
| Glutamine | 0.36 | 0.01 |
| Propionylglycine | 0.35 | 0.01 |
| Glycine | 0.35 | 0.01 |
| Gamma-glutamylglycine | 0.32 | 0.01 |
| 5-oxoproline | 0.30 | 0.02 |
| Alpha-ketoglutaramate* | 0.28 | 0.03 |
| Folate | 0.28 | 0.03 |
| N-formylanthranilic acid | 0.27 | 0.04 |
| Adenosine | −0.30 | 0.02 |

Adrenoylcarnitine
(C22:4)*

| | | |
|---|---|---|
| Arachidonoylcarnitine | 0.93 | 8.00E−26 |
| (C20:4) | | |
| Docosapentaenoylcarnitine | 0.85 | 8.64E−18 |
| (C22:5n3)* | | |
| Eicosenoylcarnitine | 0.74 | 2.35E−11 |
| (C20:1)* | | |
| Palmitoylcarnitine (C16) | 0.70 | 8.13E−10 |
| Myristoylcarnitine (C14) | 0.69 | 2.17E−09 |
| Laurylcarnitine (C12) | 0.49 | 8.88E−05 |
| Fructose | 0.41 | 1.20E−03 |
| N-acetylasparagine | 0.38 | 2.60E−03 |
| Stearoylcarnitine (C18) | 0.31 | 0.02 |
| Methionine | 0.26 | 0.04 |
| Cys-Gly | 0.26 | 0.05 |
| Arachidoylcarnitine (C20)* | 0.26 | 0.05 |
| N-palmitoylserine | 0.25 | 0.05 |
| fCysteine/fCystine | −0.29 | 0.03 |
| fCystine | −0.30 | 0.02 |

TABLE 17
Correlation coefficients between the five core metabolite
model from Table 6 that provide the highest accuracy.

| Metabolites | Correlation Coefficient | P-value |
|---|---|---|
| Glu-Cys × Histidylglutamate | −0.01 | 0.92 |
| Glu-Cys × Cinnamoylglycine | −0.06 | 0.63 |
| Glu-Cys × Proline | −0.09 | 0.51 |
| Glu-Cys × Adrenoylcarnitine | 0.01 | 0.95 |
| (C22:4)* | | |
| Histidylglutamate × | −0.03 | 0.81 |
| Cinnamoylglycine | | |
| Histidylglutamate × Proline | −0.07 | 0.59 |
| Histidylglutamate × | −0.06 | 0.65 |
| Adrenoylcarnitine (C22:4)* | | |
| Cinnamoylglycine × Proline | 1.80E−03 | 0.99 |
| Cinnamoylglycine × | −0.13 | 0.31 |
| Adrenoylglycine (C22:4)* | | |
| Proline × Adrenoylcarnitine | 0.04 | 0.74 |
| (C22:4)* | | |

Most of the metabolites listed in Tables 1 and 2 that were significantly different between the ASD and TD groups were found to be significantly correlated with the 5 core metabolites. However, there were 5 metabolites that were significantly different between the ASD-M and TD-M groups that did not significantly correlate with the 5 core metabolites. These five metabolites were B12, cis-4-decenoylcarnitine (010:1), catechol sulfate, 7-methylxanthine, and tiglylcarnitine (05:1-D0). A correlation analysis was conducted to determine if any of the 5 metabolites were correlated with one another, possibly forming a 6th group of correlated metabolites. However, none of the 5 metabolites were significantly correlated with one another. So, it appears that there are 5 primary sets of metabolites, and 5 additional metabolites that are not part of those 5 groups that are significantly different between the ASD-M and TD-M groups.

### Carnitine and Beef.

Since the levels of carnitine-conjugated molecules were lower in the ASD-M group (see Table 4), and since beef is the primary dietary source of carnitine (some can also be made by the body), hypothesis testing was performed on the beef quantity and beef frequency in the mother's diets to see if there was a difference between the two groups of mothers. The results are shown below.

TABLE 18
Univariate hypothesis testing results for
beef intake of mothers during pregnancy.

| | | | | | Ratio |
|---|---|---|---|---|---|
| | | | | | (ASD-M/ |
| Variable | Test | p-Value | FDR | AUC | TD-M) |
| Beef Frequency | MW | 0.73 | 1.00 | 0.53 | 1.12 |
| Beef Quantity | MW | 0.83 | 1.00 | 0.51 | 1.41 |

There was no significant difference found in the mean/median of the beef consumption frequency and quantity between the two groups. Also, the beef consumption frequency and quantity measurements did not significantly correlate with carnitine levels, except for a slight negative correlation of beef frequency and lignoceroylcarnitine (C24) (r=−0.26, p=0.05, unadjusted).

### Discussion

### Univariate Analysis.

Univariate hypothesis testing was performed to determine if there were significant differences between the two groups of mothers for each metabolite. The first set of univariate hypothesis testing involved the metabolites from the FOCM/TS pathways, additional nutritional information, and MTHFR gene information. These hypothesis tests revealed that only five of these measurements have a significant difference in the mean/median between the two groups of mothers (see Table 10). Hypothesis testing was next performed on the 50 metabolites from Metabolon with the highest AUC. Forty-five of these 50 metabolites were found to have significant differences (p≤0.05, FDR≤0.1) between the two groups of mothers. Additionally, three other metabolites, not found among the 50 with the highest AUC, also showed statistically significant differences between the two groups (see Table 20). This reveals that, in addition to abnormalities in the FOCM/TS pathway previously identified, there are also many other metabolic pathway differences between mothers of children with/without ASD.

Table 12 lists the significantly different metabolites by pathway, with the primary categories being amino acids, carnitines, and xenobiotics. In almost all cases these particular metabolites were significantly lower in the ASD-M group. This does not appear to be an artifact of the study, because all samples were collected identically and processed and analyzed together, and most metabolites were not significantly different between the ASD-M and TD-M groups. So, the large number of metabolites listed in Tables 11 and 12 suggest that there are in fact many metabolic differences between the ASD-M and TD-M groups.

### Multivariate Analysis.

FOCM/TS. Multivariate analysis was performed to investigate if the metabolites measured would be able to classify a mother as either having had a child with ASD (ASD-M) or a typically-developing child (TD-M). When using just the metabolites from the FOCM/TS metabolites, a combination of five metabolites appeared to have the lowest misclassification errors calculated using leave-one-out cross-validation. These metabolites included tCysteine, Glu-Cys, fCysteine, fCystine/fCysteine, and Nitrotyrosine. The Type I/Type II errors were approximately 24% and 27%. These errors show that the first subset of metabolites have only modest ability to classify the two groups of mothers.

It is interesting to note that the present results for the FOCM/TS analysis revealed substantially less ability to distinguish the ASD mothers than a similar study. The key difference is that the present example analyzed FOCM/TS metabolites 2-5 years after birth, whereas the other study evaluated mothers during pregnancy; in other words, measurements during pregnancy were better predictors of ASD risk.

FOCM/TS plus nutritional information and MTHFR. The addition of other biomarkers (B12, Folate, Ferritin, MMA, Vitamin E, and MTHFR) to the FOCM/TS metabolites did not significantly improve classification with either FDA or logistic regression.

Full set of measurements. The fourth subset of metabolites included the FOCM/TS metabolites, the nutritional biomarkers, the MTHFR gene information, and 50 metabolites from the 600 metabolites measured by Metabolon. Since there were such a large number of measurements from Metabolon, the 50 metabolites with the highest AUC were included in the analysis. This resulted in a total of 77 measurements (50 from the Metabolon data, 20 from FOCM/TS, 5 nutritional biomarkers, and MTHFR information) used for classification. Using this larger set of information, the classification errors decreased significantly. The best combination of five metabolites was found to have misclassification errors as low as 3%. This combination included one metabolite from the FOCM/TS metabolites (Glu-Cys). At least for this study, the metabolites of the FOCM/TS pathway provide some information for a modest classification but other metabolites play an even more important role. Correlation analysis (Table 9) revealed that there appear to be 5 primary categories of significantly different metabolites, with significant correlations within the group to the primary metabolite, but low correlations between the 5 primary metabolites. Almost all of the metabolites which were significantly different between the ASD-M and TD-M groups (see Tables 16 and 17) fell into 1 of these 5 groups. However, there were 5 metabolites that did not significantly correlate with any of the primary metabolites and did not correlate with each other.

### Carnitine-Conjugated Metabolites.

The univariate analysis found that all but one carnitine-conjugated metabolite (Adrenoylcarnitine (C22:4)*) were significantly lower in the ASD-M group, with the ratio of carnitine levels for ASD-M/TD-M ranging from 0.66 to 0.87, with an average of 0.78. Carnitine can be produced by the body, but there is some dietary intake also, with the only common dietary sources of carnitine being beef and (to a lesser extent) pork. There were no significant differences in the beef consumption quantity and frequency between the two groups of mothers. This suggests a metabolic difference in the production/usage of carnitine between the two groups leading to lower carnitine levels in the ASD-M group.

### Implication on Possible Role of Nutritional/Metabolic Status as a Risk Factor for ASD, and Possible Effect of Improving Nutritional/Metabolic Status on Reducing Risk of ASD.

Couples who have had a child with ASD have an 18.7% chance of future children being diagnosed with ASD, while the general risk for ASD is approximately 1.7%. Results of the analysis presented herein indicate that measurements of Glu-Cys, histidylglutamate, cinnamoylglycine, proline, and adrenoylcarnitine (C22:4)* may be able to predict with approximately 97% accuracy whether a woman, while she is not pregnant, had a child with ASD in the previous 2-5 years. Furthermore, these results suggest that therapies to address these metabolic differences may be worth investigating for decreasing the differences between metabolite levels in the two groups and potentially even reducing the risk of having a child with ASD. Metabolites of the FOCM/TS pathway are abnormal in the ASD-M group. Furthermore, a meta-analysis of 12 studies had found that supplementation with folic acid during pregnancy results in a significantly reduced risk of ASD in the children, with some studies suggesting that folic acid supplementation during the first two months of pregnancy is most important. Levels of folate were non-significantly lower in the ASD-M group in this study (17% lower, p=0.20, n.s.), but folate levels were significantly correlated with two of the 5 key metabolites (Glu-Cys and proline). Similarly, vitamin B12 levels were significantly lower in the ASD-M group, and significantly correlated with 6 of the top 50 metabolites, and abnormal maternal levels of vitamin B12 may be associated with an increased risk of ASD. Vitamin B12 and folate work together in recycling of homocysteine to methionine, a key step of the FOCM/TS pathway. Based upon these results, it is possible that appropriate supplementation with vitamin B12 and folate before and/or during pregnancy may help reduce the risk of ASD.

Similarly, the results of this study suggest that low levels of maternal carnitine may be correlated with the likelihood of having a child with ASD. Based upon the present findings, it is worth investigating if carnitine supplementation in pregnant women with low levels of carnitine may reduce their risk of having a child with ASD. Further analysis of the abnormal metabolic pathways investigated here may suggest other nutritional and/or pharmaceutical interventions that could lower the differences found in the two groups of mothers.

### CONCLUSIONS

In conclusion, this study found many significant differences in metabolites of mothers of children with ASD compared to mothers of typically-developing children. A subset of five metabolites was sufficient to differentiate the two groups with approximately 97% accuracy, after leave-one-out cross-validation. Almost all of the metabolites that were significantly different between the two groups were correlated with one of these five metabolites, suggesting that there are at least five areas of metabolic differences between the ASD-M group and the TD-M groups, represented by five metabolites (Glu-Cys, histidylglutamate, cinnamoylglycine, proline, adrenoylcarnitine (C22:4)*) which each correlated with many others. The results of this pilot study may be useful for guiding future studies of metabolic risk factors during conception/pregnancy/lactation.

What is claimed is:

1. A method for determining maternal risk of a female subject bearing a child with Autism Spectrum Disorder (ASD), the method comprising measuring the level of one or a combination of two or more metabolites selected from the metabolites listed in Table 1, Table 9, and Table 10 in a biological sample obtained from the subject, wherein a level of the one or combination of metabolites in the biological sample significantly different from the level of the one or combination of metabolites in a control panel of metabolite levels is indicative of a risk of having a child with ASD.

2. The method of claim 1, wherein the one or more metabolites are measured by preparing a sample extract and using Ultrahigh Performance Liquid Chromatography-Tandem Mass Spectroscopy (UPLC-MS/MS) to obtain the levels of the one or the combination of two or more metabolites in the reconstituted sample extract.

3. The method of claim 2, wherein the sample extract is prepared by subjecting the sample to methanol extraction.

4. The method of claim 3, wherein a dried sample extract is prepared from the methanol extraction.

5. The method of claim 4, wherein the dried sample extract is reconstituted for measuring the level of the one or combination of two or more metabolites.

6. The method of claim 1, wherein a significantly different level of the one or combination of metabolites is determined by applying each of the measured levels of the metabolites against a control panel of metabolite levels created by measuring metabolite levels of the one or combination of metabolites in control subjects with no history of bearing a child with ASD.

7. The method of claim 6, wherein the panel is stored on a computer system.

8. The method of claim 6 wherein the applying comprises:
a. when the level of one metabolite is measured,
i. comparing the measured level of the metabolite in the sample to the level of the metabolite in the control panel of metabolite levels using a statistical analysis method selected from the standard Student t-test, the Welch test, the Mann-Whitney U test, the Welch t-test, and combinations thereof; and
ii. calculating the false discovery rates (FDR) and optionally the false positive rate (FPR) for the metabolite; and
b. when the levels of a combination of two or more metabolites are measured, calculating the Type I (FPR) and Type II (FNR) errors for the combination of metabolites using FDA or logistic regression.

9. The method of claim 8, wherein:
a. when the level of one metabolite is measured, a p-value of less than or about 0.05 and an FDR value is less than or about 0.1, is indicative of a risk of having a child with ASD; and
b. when the levels of a combination of two or more metabolites are measured, a Type I error of about or below 10% and a Type II error of about or below 10% is indicative of a risk of having a child with ASD.

10. A method for determining increased maternal risk of a female subject bearing a child with ASD, the method comprising:
a. obtaining or having obtained a biological sample from the female subject;
b. subjecting the sample to methanol extraction;
c. drying the sample extract;
d. reconstituting the sample extract;
e. measuring the level of one or a combination of two or more metabolites selected from the metabolites listed in Table 1, Table 9, and Table 10 in the reconstituted sample extract using Ultrahigh Performance Liquid Chromatography-Tandem Mass Spectroscopy (UHPLC-MS/MS),
f. applying each of the measured levels of the metabolites against a control panel of metabolite levels created by measuring metabolite levels of the one or combination of metabolites in control subjects with no history of bearing a child with ASD, wherein the panel is stored on a computer system and wherein the applying comprises:
i. when the level of one metabolite is measured,
1. comparing the measured level of the metabolite in the sample to the level of the metabolite in the control panel of metabolite levels using a statistical analysis method selected from the standard Student t-test, the Welch test, the Mann-Whitney U test, the Welch t-test, and combinations thereof; and
2. calculating the false discovery rates (FDR) and optionally the false positive rate (FPR) for the metabolite;
ii. when the levels of a combination of two or more metabolites are measured, calculating the Type I (FPR) and Type II (FNR) errors for the combination of metabolites using FDA or logistic regression;
g. indicating that the female subject has an increased risk of bearing a child with ASD if:
i. when the level of one metabolite is measured, the level of the metabolite in the biological sample is significantly different from the level of the metabolite in the control panel of metabolite levels if the p-value is less than or about 0.05 and the FDR value is less than or about 0.1; and
ii. when the levels of a combination of two or more metabolites are measured, the Type I error is about or below 10% and the Type II error is about or below 10%.

11. The method of any one of the preceding claims, wherein the biological sample comprises any one of synovial, whole blood, blood plasma, serum, urine, breast milk, and saliva.

12. The method of any one of the preceding claims, wherein the biological sample comprises cells.

13. The method of any one of the preceding claims, wherein the biological sample is whole blood.

14. The method of any one of the preceding claims, further comprising removing protein from the sample extract.

15. The method of any one of the preceding claims, wherein the level of a metabolite is measured using:
i. reverse phase chromatography positive ionization methods optimized for hydrophilic compounds (LC/MS Pos Polar);
ii. reverse phase chromatography positive ionization methods optimized for hydrophobic compounds (LC/MS Pos Lipid);
iii. reverse phase chromatography with negative ionization conditions (LC/MS Neg); and
iv. a HILIC chromatography method coupled to negative (LC/MS Polar).

16. The method of any one of the preceding claims, wherein the level of a metabolite is calculated from a peak area and standard calibration curve obtained for the metabolite using the UPLC-MS/MS.

17. The method of any one of the preceding claims, wherein measuring further comprises identifying each metabolite by automated comparison of the ion features in the sample extract to a reference library of chemical standard entries that included retention time, molecular weight (m/z), preferred adducts, and in-source fragments as well as associated MS spectra.

18. The method of any one of the preceding claims, wherein the method further comprises calculating the area under the curve (AUC) of the receiver operating characteristic (ROC) curve for each metabolite.

19. The method of any one of the preceding claims, wherein the risk is determined pre-conception, during pregnancy, or after giving birth to the child.

20. The method of any one of the preceding claims, wherein the risk is determined pre-conception.

21. The method of any one of the preceding claims, wherein the risk is determined during pregnancy.

22. The method of any one of the preceding claims, wherein the risk is determined after giving birth to the child.

23. The method of any one of the preceding claims, wherein the control panel comprises metabolite levels measured in biological samples obtained from mothers of children lacking any clinical indicators of ASD.

24. The method of any one of the preceding claims, wherein the level of one metabolite is measured.

25. The method of claim 24, wherein the metabolite is selected from the metabolites listed in Table 2 and Table 10.

26. The method of claim 24, wherein the metabolite is Histidylglutamate or N-acetylasparagine.

27. The method of one of claims 1-23, wherein the levels of a combination of two metabolites are measured.

28. The method of claim 27, wherein the two metabolites are selected from the combinations of metabolites listed in Table 3 and Table 14.

29. The method of claim 27, wherein the two metabolites are N-acetylasparagine and X-12680.

30. The method of claim 27, wherein the two metabolites are Histidylglutamate and 6-hydroxyindoel sulfate.

31. The method of one of claims 1-23, wherein the levels of a combination of three different metabolites are measured.

32. The method of claim 31, wherein the three metabolites are selected from the combinations of metabolites listed in Table 4 and Table 14.

33. The method of claim 31, wherein the three metabolites are 6-hydroxyindole sulfate, histidylglutamate, and N-acetylasparagine.

34. The method of claim 31, wherein the three metabolites are 6-hydroxyindole sulfate, histidylglutamate, and N-acetylasparagine.

35. The method of claim 31, wherein the three metabolites are histidylglutamate, N-acetylasparagine, and X-21310.

36. The method of claim 31, wherein the three metabolites are 3-indoxyl sulfate, histidylglutamate, and N-acetylasparagine.

37. The method of claim 31, wherein the three metabolites are Histidylglutamate, N-formylanthranilic acid, and palmitoylcarnitine (C16).

38. The method of one of claims 1-23, wherein the level of a combination of four metabolites is measured.

39. The method of claim 38, wherein the four metabolites are selected from the combination of metabolites in Table 5 and Table 14.

40. The method of claim 38, wherein the four metabolites are Histidylglutamate, S-1-pyrroline-5-carboxylate, N-acetyl-2-aminooctanoate*, and 5-methylthioadenosine (MTA).

41. The method of one of claims 1-23, wherein the level of a combination of five metabolites is measured.

42. The method of claim 41, wherein the five metabolites are selected from the combination of metabolites in Table 6 and Table 15.

43. The method of claim 41, wherein the five metabolites are Glu-Cys, histidylglutamate, cinnamoylglycine, proline, and adrenoylcarnitine (C22:4)*.

44. The method of claim 43, wherein each metabolite represents a group of metabolites correlated with the metabolite, and wherein metabolites correlated with each metabolite is listed in Table 16.

45. The method of claim 44, wherein the levels of metabolites correlated with each metabolite are also measured.

46. The method of any one of the preceding claims, wherein the method determines the maternal risk of bearing a child with ASD with a sensitivity of at least about 80%, a specificity of at least about 80%, or both.

47. The method of any one of the preceding claims, wherein the method determines the maternal risk of bearing a child with ASD with a sensitivity of at least about 90%, a specificity of at least about 90%, or both.

48. The method of any one of the preceding claims, wherein the method determines the maternal risk of bearing a child with ASD with a misclassification error of about 3%.

49. The method of any one of the preceding claims, wherein the method determines the maternal risk of bearing a child with ASD with with an accuracy of about 95% or more.

50. The method of any one of the preceding claims, further comprising assigning a medical, behavioral, and/or nutritional treatment protocol to the subject when the subject is at increased risk of bearing a child with ASD.

51. The method of claim 50 wherein assigning a medical, behavioral, and/or nutritional treatment protocol to the subject comprises assigning one or more treatment protocols personalized to the subject.

52. The method of claim 51, wherein the treatment protocol comprises adjusting the level of one or a combination of two or more metabolites in the subject.

53. The method of claim 52, wherein the metabolite or combination of two or more metabolites are selected from the one or combination of two or more metabolites identified as having a level in the biological sample significantly different from the level of the one or combination of metabolites in the control sample.

54. The method of claim 53, wherein the metabolite is a metabolite associated with the one or combination of two or more metabolites identified as having a level in the biological sample significantly different from the level of the one or combination of metabolites in the control sample.

55. The method of claim 51, wherein the treatment protocol comprises supplementation with vitamin B12 and folate before and/or during pregnancy.

56. The method of any one of the preceding claims, further comprising assigning a medical, behavioral, and/or nutritional treatment protocol to a child born to a subject determined to be at high risk of having a child with ASD.

57. The method of claim 56, wherein assigning a medical, behavioral, or and/or nutritional treatment protocol to the child comprises assigning one or more treatment protocols personalized to the child.

58. A method of determining a personalized treatment protocol for a pregnant subject or a subject contemplating conception and at risk of having a child with ASD, the method comprising measuring in a biological sample obtained from the subject the level of one or combination of two or more metabolites selected from the metabolites listed in Table 1, Table 9, and Table 10 and any combination thereof, identifying one or a combination of metabolites having a level in the biological sample significantly different from the level of the one or combination of metabolites in a control sample, and assigning a personalized medical, behavioral, or nutritional treatment protocol to the subject.

59. A method of monitoring the therapeutic effect of an ASD treatment protocol in a pregnant subject or a subject contemplating conception and at risk of having a child with ASD, the method comprising measuring in a first biological sample obtained from the subject the level of one or a combination of metabolites selected from the metabolites listed in Table 1, Table 9, and Table 10 and any combination thereof, measuring in a second biological sample obtained from the subject the level of the one or combination of metabolites, and comparing the level of the one or combination of metabolites in the first sample and the second sample, wherein maintenance of the level of the one or combination of metabolites or a change of the level of the one or combination of metabolites to a level of the one or combination of metabolites in a control sample is indicative that the treatment protocol is therapeutically effective in the subject.

60. The method of claim 58 or 59, wherein the treatment protocol comprises supplementation with vitamin B12 and folate before and/or during pregnancy may help reduce the risk of ASD.

61. A kit for performing the method of any one of claim 1, 9, 58 or 59, the kit comprising: (a) a container for collecting the biological sample from the subject; (b) solutions and solvents for preparing an extract from a biological sample obtained from the subject; and (c) instructions for (i) preparing the extract, (ii) measuring the level of one or more metabolites selected from the metabolites listed in Table 1, Table 9, and Table 10 using Ultrahigh Performance Liquid Chromatography-Tandem Mass Spectroscopy (UPLC-MS/MS); and (iii) applying the measured metabolite levels against a control panel of metabolite levels created by measuring metabolite levels of the one or combination of metabolites in control subjects with no history of bearing a child with ASD.
